(19) United States
(12) Patent Application Publication
(10) Pub. No.: US 2007/0185231 A1
(43) Pub. Date: Aug. 9, 2007

(54) BONE CEMENT COMPOSITE CONTAINING PARTICLES IN A NON-UNIFORM SPATIAL DISTRIBUTION AND DEVICES FOR IMPLEMENTATION

(76) Inventors: Y. King Liu, Petaluma, CA (US); Jan R. Lau, Windsor, CA (US); John Stalcup, Glen Ellen, CA (US); Michael T. Lyster, Riverwoods, IL (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/626,336

(22) Filed: Jan. 23, 2007

Related U.S. Application Data
(60) Provisional application No. 60/761,454, filed on Jan. 23, 2006.

(51) Int. Cl. A61K 6/08

(52) U.S. Cl. 523/116

(57) ABSTRACT

One embodiment of the invention comprises a differential composite in which bone cement everywhere or substantially everywhere contains at least some non-zero volume fraction of particles, and in which the local volume fraction of particles may vary from place to place in the composite in a controlled manner. The variation may be by identifiable region or may be in the form of a gradient of the local volume fraction of particles. In at least some places, the local volume fraction of particles may be such that the particles act as crack arrestors. Close to the interface with natural bone, the local volume fraction of particles may be greater. In at least some places adjoining natural bone, the local volume fraction of particles may be such as to allow bone ingrowth into appropriate region(s) of the composite, resulting in improved interfacial shear strength. Methods and apparatuses for producing and delivering the composite are also disclosed, which may include use of an introducer and an expandable basket-type device.

### CROSS-REFERENCE TO RELATED APPLICATIONS

This application claims priority under 35 U.S.C. § 119 to U.S. Provisional Application No. 60/761,454, filed Jan. 23, 2006, the disclosure of which is incorporated by reference herein in its entirety.

### BACKGROUND OF THE INVENTION

The invention relates to bone cement, and more specifically to bone cement that contains particles for the purpose of improving its biomechanical and biomaterial properties. This invention further relates to devices and methods for its implementation, which are also disclosed herein.

Bone cement has often been used as grout to provide support either for implantation of prostheses or for reduction of diseased bone. Bone cement has been widely used in artificial total joint replacements in orthopedic surgery in order to bond implants to bone.

One formulation is to mix the powder and monomer of polymethylmethacrylate (PMMA) until it reaches a “dough-like” stage and then is inserted, often under pressure, into a previously prepared cancellous bone cavity. The PMMA flows into the cavity as well as all the potential interstitial spaces of the cancellous bone matrix. The stem of the prosthesis is then inserted into the dough-like PMMA. In a few minutes, the PMMA hardens, thus securing the metallic orthopedic surgical implants. While this has been somewhat satisfactory, such cement is subjected to the cyclic load-bearing of daily living and, thus, to corrosion fatigue. “Penny” cracks form naturally because the cement partially surrounding the cancellous bone matrices are crack initiators. Once initiated, cracks can propagate through the solidified polymer. FIG. 1 presents data showing crack initiation and its growth rate as a function of stress intensity factor for pure PMMA cement (i.e., the curve represented by the triangle data points on the left side of the FIG. 1). For this reason, implants, affixed using such bone cement, have sometimes experienced mechanical failures, after an average of 10 years, requiring another surgery, e.g., a revision repair.

It is also known that composite cement, which contains particles mixed in with the cement, act as crack arrestors. FIG. 1 further exhibits data about crack growth rate as a function of stress intensity factor for composite PMMA, which contains 30% by weight of inorganic bone particles sized between 150 to 300 microns (i.e., the curve represented by the cross data points on the right side of FIG. 1). The data shows that the presence of the particles resulted in a crack growth rate that was about one order of magnitude slower than the crack growth rate for the corresponding pure solidified PMMA polymer.

Another relevant parameter is the shear strength of the interface between the bone and the cement. Experimental canine tests of bone-bone-particle impregnated bone cement interfaces have shown that its interfacial shear strength between bone and composite bone cement is improved by a factor of 3.6, after 5 months, when compared to the interfacial shear strength between bone and PMMA cement. Histology and contact radiography confirmed bone ingrowths into the interfacial spaces between the natural bone and the bone-particle impregnated bone cement. These findings were obtained for cements, which contained particles distributed throughout the cement region. The distribution of particles in the cement may have been slightly non-uniform because of the method of mixing the PMMA polymer powder with particles and the PMMA monomer in the normal doughing process, but any such non-uniformity was uncontrolled. These findings were described in: Y. K. Liu, J. B. Park, G. O. Njus, and D. Stienstra, “Bone-particle-impregnated bone cement: An in vitro study,” Journal of Biomedical Materials Research, Vol. 21, 247-261, 1987; H. C. Park, Y. K. Liu and R. S. Lakes, “The material properties of bone-particle impregnated PMMA,” Journal of Biomechanical Engineering, Vol. 108, 141-148, 1986; and in K. R. Dai, Y. K. Liu, J. B. Park, C. R. Clark, K. Nishiyama, Z. K. Zheng, “Bone-particle-impregnated bone cement: An in vivo weight-bearing study,” Journal of Biomedical Materials Research, Vol. 25, 141-156, 1991.

For the exemplary situation of cement implantations in vertebroplasty or kyphoplasty, the use of particle-containing PMMA cement, with particles distributed throughout the cement acting as crack-arrestors, may mitigate corrosion fatigue failure of the “pure” particle-free PMMA. Such low-volume implants may fail due to another cause, that is, interfacial shear-stress failure at the bone-bone cement interface because of sparse particle-bone cement contact resulting in little or no bone ingrowth. The interfacial shear-stress failure becomes acute when the volume of the cement composite is decreased, e.g., the number of inter-touching particles at the surface of the cement composite is lessened in vertebroplasty and kyphoplasty. Thus, there is room for improvement in regard to both the fatigue life in the cement bulk and interfacial shear strength of the bone-cement interface, respectively.

In other works, U.S. Pat. No. 5,343,877 to Park and U.S. Pat. No. 5,681,317 to Caldarise, both of which are hereby incorporated by reference in their entirety, disclose a bone-cemented joint in which the region near the bone contains particles, but the bulk region of the bone cement contains no particles. These techniques were intended for use in implanting metallic stems of hip and knee prostheses with the particle-containing region bordering natural bone. In these usages, the region in which the particle-impregnated cement was applied would receive a minimal crack-arresting benefit in the thin layer already discussed, but the cement-only (particle-free) interior bulk region would have the same vulnerability to corrosion fatigue as was found for substantially pure solidified PMMA cement. The probability of bone ingrowth between the bone and bone-cement interface is very likely to be low because of the small amount of cement volume utilized.

In regard to surgery not involving metallic implants, e.g., surgical procedures known as vertebroplasty and kyphoplasty that involve injection of bone cement into either cancellous bone with or without a cavity within an individual intervertebral joint, U.S. Pat. No. 6,332,894 to Stalcup, which is herein incorporated by reference in its entirety, discloses a method to fuse two vertebrae by injecting cement, which may contain bone particles, into an annular balloon between vertebrae to achieve fusion of the intervertebral motion segment without internal fixation with instrumentation.

It would be desirable to provide an improved bone cement system, having both optimal crack arresting and interfacial shear strength characteristics, for implantation of prostheses, for repair of vertebrae and other bones, and for any other appropriate surgical procedure.

It would further be desirable to provide apparatus and methods for creating and dispensing such a composite.

### SUMMARY OF THE INVENTION

An embodiment of the invention comprises a composite within or in contact with bone, in which the composite everywhere contains bone cement which contains at least some non-zero volume fraction of particles (which may be biocompatible and bioresorbable). In this composite, the local volume fraction of particles may vary from place to place in the composite in a controlled manner. The variation may be by identifiable region or may be in the form of a gradient of the local volume fraction of particles. In at least some places, the local volume fraction of particles may be such that the spaces created by the particles act as crack arrestors. At places that are close to the interface with natural bone, the local volume fraction of particles may be greater. In at least some places adjoining natural bone, the local volume fraction of particles may be such as to allow bone ingrowth into appropriate regions of the composite, resulting in much improved interfacial shear strength. An embodiment of the invention also includes methods and apparatus for producing and dispensing this differential configuration of composite, which may include use of a cannula introducer and a deployable double-umbrella basket.

In one aspect, disclosed is a composite that includes bone cement, and particles contained in said bone cement. The composite can have at least two different non-zero local particle concentrations of the particles. The local particle concentrations can be controlled to have desired values at desired locations. In some aspects, the composite immediately adjacent to a bone has a particle concentration greater than a particle concentration away from the bone. In some aspects, the bone cement is substantially non-bioresorbable, or can be substantially bioresorbable. The composite can be contained within one or more cavities in one or more bones of a patient. The composite can exhibit a gradient of the local concentration of the particles.

In some embodiments, the particles can include at least one of the following substances: inorganic bone; demineralized bone; natural bone; bone morphogenic protein; collagen; gelatin; polysaccharides; polycaprolactone (PCL); polyglycolide (PGA); polylactide (PLA); DLPLG which is a copolymer of PLA and PGA; polyparadioxanone (PPDO); other aliphatic polyesters; polyphosphoester; polyphosphazenes; polyanhydrides; polyhydroxybutyrate; polyaryetherketone; polyurethanes; magnesium ammonium phosphate; strontium-containing hydroxyapatite; beta tricalcium phosphate; other forms of calcium phosphate; and carbon. In some embodiments, the bone cement includes one or more of the following substances: polymethylmethacrylate (PMMA); hydroxyethyl methacrylate (HEMA); polyalkanoate; polyetherurethane; polycarbonate urethane; polysiloxaneurethane; and polyfluoroethylene.

In some aspects, also disclosed is a composite that includes a first region including bone cement containing a first non-zero concentration of first particles, and a second region including bone cement containing a second non-zero concentration of second particles. The second concentration of second particles can be different from the first concentration of the first particles, and the regions can be controlled to occupy desired locations. In some aspects, the first region touches a bone and the second region substantially does not touch the bone. The first concentration of the first particles can be larger than the second concentration of the second particles. In some aspects, the first particles and the second particles are substantially identical to each other. In other aspects, the first particles and the second particles are different from each other in some respect. In some embodiments, also included in the composite are additional first particles which are only partially contacted by the bone cement, or even not contacted by the bone cement. In some embodiments, also included in the composite is a gradient-containing region between the first and second regions.

In some embodiments, disclosed is a composite including bone cement and particles contained in the bone cement. The composite can have at least some places that are within no more than about 2 mm of a bone. The composite can also have a local weight fraction of particles of at least about 60%. The composite more than about 2 mm away from the bone can have a local weight fraction of particles that is no more than about 40%.

In some other aspects, the composite can have at least some places that are within approximately 2 mm of a bone that are in a bone-ingrowth regime. The composite can also have places that are more than approximately 2 mm away from said bone that are in a crack-arresting regime.

In yet other aspects, any substantially equiaxial local space containing more than three of said particles there can be defined a local particle concentration, and the composite can exhibit a gradient of said local particle concentration.

In other aspects, disclosed is a stabilized vertebra or other bone that includes a vertebra or other bone having a cavity, and a composite in the cavity. The composite includes bone cement and particles contained within the cement. The particles can have different particle concentrations at different places within the composite. Also, the particle concentration immediately adjacent to bone material of the vertebra or other bone can be greater than the particle concentration away from the bone material of the vertebra. In some embodiments, the particles have different particle concentrations at different places within the composite, and the particle concentrations are controlled to have desired values at defined places.

Also disclosed herein is a method for filling a bone cavity with a particle-impregnated bone cement composite. The method includes the steps of depositing bioresorbable first particles on an interior surface of the cavity, and depositing into remaining space in the cavity a cement precursor which includes second particles. The first concentration of the first particles on the surface can be larger than a second concentration of the second particles in the cement precursor. In some embodiments, depositing the first particles includes at least one or more of the following substances: inorganic bone; demineralized bone; natural bone; bone morphogenic protein; collagen; gelatin; polysaccharides; polycaprolactone (PCL); polyglycolide (PGA); polylactide (PLA); DLPLG which is a copolymer of PLA and PGA; polyparadioxanone (PPDO); other aliphatic polyesters; polyphosphoester; polyphosphazenes; polyanhydrides; polyhydroxybutyrate; polyaryetherketone; polyurethanes; magnesium ammonium phosphate; strontium-containing hydroxyapatite; beta tricalcium phosphate; other forms of calcium phosphate; and carbon. The cement precursor can include at least one substance selected from the group consisting of: polymethylmethacrylate (PMMA); hydroxyethyl methacrylate (HEMA); polyalkanoate; polyetherurethane; polycarbonate urethane; polysiloxaneurethane; and polyfluoroethylene. In some embodiments, depositing the first particles includes depositing said first particles using an introducer and a particle-deploying device, such as an expandable basket, e.g., a double-umbrella basket. Depositing the first particles can also involve using a double-umbrella basket that includes a perforated outer membranous covering on the basket. Furthermore, depositing first particles using a double-umbrella basket can include bursting a perforated outer membranous covering on the basket. Depositing the first particles using the double-umbrella basket can also include bursting a perforated membranous covering while the covering is inside the bone cavity. In some embodiments, at any stage during the method, the additional step of causing deformation of the cavity due to pressure of expansion and/or deployment of a double-umbrella basket inside the cavity may be performed.

Also disclosed herein is an apparatus for delivering and depositing particles on a wall of a bone cavity. The apparatus can be a double-umbrella basket. The double-umbrella basket can be configured to expand by mechanical force or pressure, and when expanded releases the particles. The particles can be biocompatible and/or bioresorbable particles. The perforated outer membranous covering on the double-umbrella basket can be capable of bursting due to the pressure or force exerted on the double-umbrella basket, or alternatively the membranous covering can dissolve.

Also disclosed herein is an apparatus for delivering and depositing particles on a wall of a bone cavity. The apparatus includes a double-umbrella basket. The double-umbrella basket can be configured to expand by mechanical force or pressure, and when expanded releases the particles. In some embodiments, the particles are biocompatible and bioresorbable particles. In some embodiments, the perforated outer membranous covering on the double-umbrella basket is capable of bursting due to the pressure or force exerted on the double-umbrella basket.

Also disclosed is a method of treating a bone, including the step of introducing within the bone a cement including particles, wherein said particles comprise an osteoinductive substance. The osteoinductive substance can also be osteoconductive.

In yet another aspect disclosed is a method of treating a bone, including introducing within the bone a cement that includes particles, wherein the particles include an active pharmaceutical ingredient. The active pharmaceutical ingredient may be, e.g., an anti-neoplastic drug.

Also disclosed is a method of creating a cavity. The method includes creating an access. Next, apparatus can be introduced into the access in a first configuration. The apparatus can be expanded within the access to create the cavity. Next, the apparatus can be contracted or fully contracted within the cavity. The apparatus can then be rotated within the cavity. The apparatus can then be reexpanded within the cavity. Next, the apparatus can be fully contracted to facilitate removal of the apparatus from the cavity. Rotating the apparatus can include rotating by an angle which is less than about at least about an integer multiple of a spacing angle between adjacent struts of the apparatus. Also, the method can include the step of, after reexpanding the apparatus within the cavity, further expanding the apparatus to a greater extent.

In one aspect, disclosed is a method of creating a cavity within a bone, including the steps of creating an access hole; introducing into the access hole an apparatus in a contracted configuration having a plurality of struts comprising sharp edges; expanding the apparatus within the access hole to create the cavity; rotating the apparatus within the cavity sufficiently to cut bone within the cavity; and contracting the apparatus to facilitate removal of the apparatus from the cavity. The method can also include removing the cut bone from the cavity.

In another aspect, disclosed is an apparatus for creating a cavity within a bone, including an expandable basket having a plurality of struts. The struts can have sharp edges configured for cutting through bone.

Also disclosed herein is an apparatus for creating a cavity within a bone. The apparatus includes an expandable basket having a plurality of struts; wherein the struts have variable dimensions transverse to the long axis of the struts. In some embodiments, at least some of the struts are wider in a middle region of the struts than at end regions of the struts.

In some aspects, disclosed is an apparatus for creating a cavity within a bone. The apparatus includes an expandable basket having a plurality of struts having a first proximal end and a second distal end. Also included is a first substantially rigid member connected to or in contact with a first proximal end of said basket and a second substantially rigid member connected to or in contact with a second distal end of said basket. Moreover, the apparatus can include an assembly that includes a screw. The assembly is preferably suitable to exert an axial force on one of the members with respect to the other members. In some embodiments, the screw assembly includes a knob configured to rotate with respect to a threaded non-rotatable component. The screw assembly can receive rotation from a translational member.

Also disclosed herein is an apparatus for creating a cavity in a bone, including an expandable basket having a plurality of struts having a proximal end and a distal end; and an endcap suitable to transmit reaction force to the basket, wherein the endcap has an end which is substantially blunt. In some embodiments, the endcap further includes a shoulder region configured for the basket to bear against.

In some aspects, disclosed is an apparatus for creating a cavity within a bone, including an expandable basket having a plurality of struts having a first end and a second end, and an endcap suitable to transmit force to said basket. The endcap can have a diameter that is less than an outside diameter of the distal end of the expandable basket.

Also disclosed herein is an apparatus for creating a cavity, including an expandable basket having a plurality of struts, and a flexible membranous covering configured to move outward when the struts move outward. In some embodiments, when the apparatus is in an undeployed configuration, the membranous covering is folded inward to create a space configured to contain particles. In some embodiments, the particles can include materials which are at least osteoconductive or osteoinductive or both. In some embodiments, the membranous covering is continuous around a circumference of the apparatus. In some embodiments, the membranous covering is interspersed around a circumference of the apparatus. In some embodiments, also included is an outer membranous covering configured to either rupture or dissolve. In other embodiments, the outer membranous covering also includes holes or slits. The struts can have spaces configured to contain particles.

In some aspects, also disclosed is an apparatus for creating a cavity, including an elastomer which expands in a radial direction when said elastomer is axially compressed; and means for axially compressing said elastomer. In some embodiments, the first particles are radioopaque and the second particles are not radioopaque.

Further, disclosed is a method for treating or preventing a vertebral compression fracture. The method includes the step of inserting an insertion device percutaneously into a vertebral body. Next, the method can include the step of inserting a cavity-forming device through the insertion device into an area of cancellous bone in the vertebral body. Furthermore, the method can include the step of displacing cancellous bone with the cavity-forming device to create a cavity defined by a surface of cancellous bone. Also, the method can include the step of introducing a first media into the cavity to line at least a portion of the surface thereby reducing the volume of the cavity; and filling at least a portion of the cavity with a second media.

In another aspect, disclosed is a method for treating or preventing a vertebral compression fracture. The method can include the step of inserting an insertion device percutaneously into a vertebral body. Next, the method can include the step of inserting a cavity-forming device through the insertion device into an area of cancellous bone in the vertebral body. Next, the step of displacing cancellous bone with the cavity-forming device to create a cavity defined by a surface of cancellous bone can be performed. Further, the step of introducing a first media into the cavity to line at least a portion of the surface thereby reducing the volume of the cavity; and filling at least a portion of the cavity with a second media can also be performed. The insertion device can be a needle, which can be an eleven-gauge needle in some embodiments. The cavity-forming device can be, in some embodiments, selected from the group consisting of a mechanical tamp, a reamer, a drill, a hole puncher, and a balloon catheter. Introducing a first media into the cavity can include introducing a powder, paste, and/or suspension into the cavity.

In some aspects, also disclosed is a method of treating a bone. A cavity can be created within cancellous bone. At least a portion of the cavity can be lined with a first media. At least a portion of the cavity can be filled with a second media. The first media can include a cancellous bone ingrowth characteristic. The second media can include a crack propagation inhibitor characteristic.

In some aspects, also disclosed is a method of treating or preventing a vertebral compression fracture. A cavity can be created within cancellous bone of a vertebral body. At least a portion of the cavity can be lined with a first media. At least a portion of the cavity can be filled with a second media to form a construct having an outer surface and a core. The material of the outer surface can include a relatively greater bone ingrowth characteristic than the material of the core. In other embodiments, the material of the core has a relatively greater resistance to crack propagation than the material of the outer surface.

Also disclosed herein is a kit for treatment of a vertebral compression fracture. The kit can include a cavity forming device, a first deployment device for deploying a first media against a wall of the cavity, and a second deployment device for deploying a second media adjacent the first media within the cavity. The kit can also additionally include a volume of the first media, and/or a volume of the second media. The second media, in some embodiments, can include the polymer and monomer of PMMA. The kit can also additionally include access tools for creating access to a vertebral body.

Various kits for treating vertebral compression fractures are disclosed. The kit can include a cavity forming device, a first deployment device for deploying a first media against a wall of the cavity, and a second deployment device for deploying a second media adjacent to the first media within the cavity. The kit can also additionally include a volume of the first media, and/or a volume of the second media. The second media, in some embodiments, can include the polymer and monomer of PMMA. The kit can also additionally include access tools for creating access to a vertebral body. In some kits, included is a drill for accessing the interior of the bone. In some kits, the cavity forming device can be, for example, an inflatable balloon or other expandable device. The kit can also include a removal tool such as a rotatable loop or cutter for cutting cancellous bone, a dispensing tool for dispensing particulate within the cavity, and/or a dispensing tool for dispensing bone cement within the particulate.

In some embodiments, a kit can include one or more of the following: a sharp incision tool such as one or more scalpels, a clamp or spreader to keep incision open, a drill to create access port in the vertebral wall or a pedicle, and an instrument to create a cavity in cancellous bone. Also included can be a cavity-creating support instrument such as an inflator if the cavity is created with a balloon and a suction device if cancellous bone material removal is required. A device for injecting bone cement, or a precursor in the cavity, and supporting instruments such as a tamp, syringe, and a device that accelerates the curing process of the cement can also be included. Some kits also include a wound closure device, such as sutures, staples, or adhesives.

### BRIEF DESCRIPTION OF THE DRAWINGS

The invention is further illustrated in the following Figures.

FIG. 1 illustrates experimental data showing the crack propagation growth rate, da/dN, as a function of stress intensity factor ΔK, for pure PMMA bone cement and also for PMMA bone cement containing 30% by weight of inorganic bone particles, which was found by Y. K. Liu, J. B. Park, G. O. Njus, and D. Stienstra, “Bone-particle-impregnated bone cement: An in vitro study,” Journal of Biomedical Materials Research, Vol. 21, 247-261, 1987 to optimize fatigue life in a standardized ambient test.

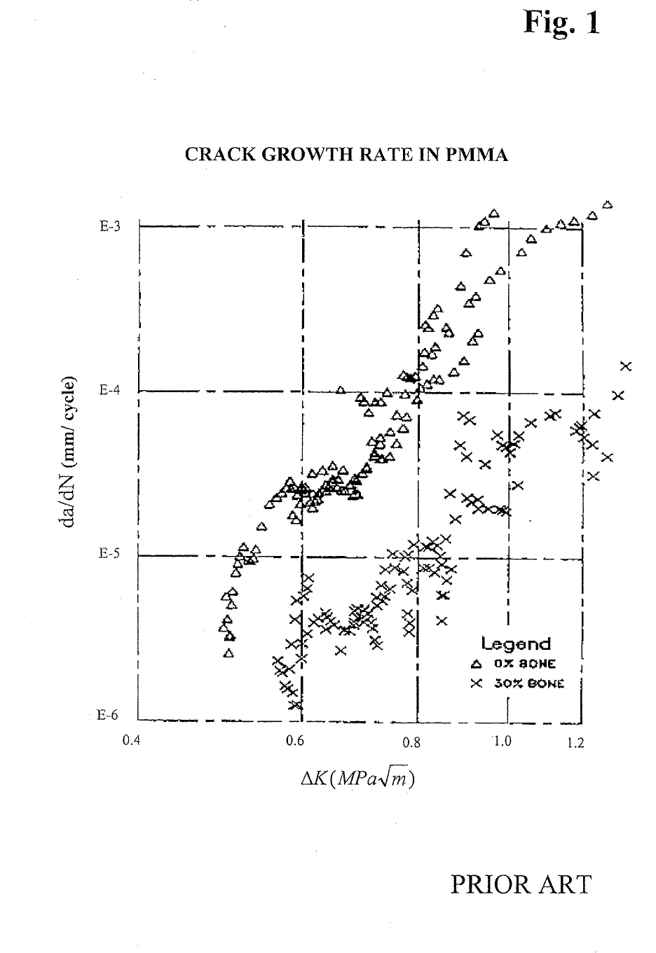

FIG. 2 illustrates a cross-section of two regions within the composite in place in a bone, according to one embodiment of the invention.

FIG. 3 further illustrates that a first region has a higher volume fraction of particles and a second region has a lower volume fraction of particles, according to one embodiment of the invention. In this illustration, the composite cement material substantially surrounds all of the particles and contacts the natural bone.

FIG. 4 illustrates the regions somewhat similarly to FIG. 3; however, this illustration shows that there are some particles which are only partially contacted or not contacted at all by the cement, according to one embodiment of the invention.

FIG. 5 is a cross-section that illustrates a composite having a gradient of local volume fraction of particles, according to one embodiment of the invention.

FIG. 6A is a cross-section that shows a notch in the pedicular wall of a vertebra created by a trocar as part of a surgical procedure, according to one embodiment of the invention.

FIG. 6B is a cross-section that shows a hole drilled with a drill bit through the pedicular wall of a vertebra into the cancellous bone of the vertebra, as part of a surgical procedure, according to one embodiment of the invention.

FIG. 6C is a cross-section that shows the hole that was drilled through the pedicular wall of a vertebra into the cancellous bone of the vertebra as part of a surgical procedure, according to one embodiment of the invention.

FIG. 6D is a cross-section that shows a cannula introducer placed into an access channel through the pedicular wall of vertebra and into the interior of the cancellous bone matrix of the vertebral body, as part of a surgical procedure, according to one embodiment of the invention.

FIG. 7 is a cross-section that shows the potential cavity of FIG. 6D after a layer of densely-packed particles is in position on the wall of the bone cavity, according to one embodiment of the invention.

FIG. 8 is similar to FIG. 7, but further shows particle-containing cement having been introduced into the interior of the region defined by the densely-packed layer of particles adjoining the wall of the potential cavity, according to one embodiment of the invention.

FIG. 9 is similar to FIG. 8, but further shows that the cement may spread beyond the space into which it was originally introduced and may be extended among the particles in the densely-packed layer of particles adjoining the wall of the potential cavity and beyond, according to one embodiment of the invention.

FIGS. 10-19 illustrate steps of a surgical procedure involving an expandable cage in the form of a deployable double-umbrella basket apparatus for delivering and depositing composite bone cement into a bone cavity, according to one embodiment of the invention. Specifically, FIG. 10 is a cross-section that shows the assembly of the cannula introducer 47 and the double-umbrella basket 48 outside the surgical site, according to one embodiment of the invention.

FIG. 11 is a cross-section that shows the assembly of the cannula introducer 47 and the double-umbrella basket 48 that may be inserted into the surgical site 150, according to one embodiment of the invention.

FIG. 12 shows a side sectional view (on the left) and an end sectional view (on the right) that shows between each strut 51 of the double-umbrella basket 48 is a contiguous or semi-continuous inner membrane covering 50 that may surround or partially surround the double-umbrella basket 48 and may hold particles 172 within the undeployed double-umbrella basket 48, according to one embodiment of the invention. The inner membrane covering 50 may fold inward in the undeployed configuration to create particle-containing reservoirs. The particles 172 are further contained by a biocompatible and bioresorbable outer membrane covering 53, which may be firmly affixed to struts 51. The outer membrane coverings 53 between struts 51 can be prefabricated with perforated tear slots 52.

FIG. 13 is a cross-section illustrating that after the assembly of the cannula introducer 47 and the double-umbrella basket 48 reaches within the surgical site 150, the cannula introducer 47 may be partially retracted exposing the undeployed double-umbrella basket 48, according to one embodiment of the invention.

FIG. 14A is a perspective view of a device that can deploy the double-umbrella basket 48 by a mechanically-threaded rod 54 when the knob 55 of the rod 54 assembly is rotated in an appropriate direction, according to one embodiment of the invention. Rotating the knob 55 of the rod 54 may move the rod 54 distally, which applies an axial force onto the basket assembly. The axial force may cause the basket assembly struts 51 to curve outward; thus, creating an end-to-end double-umbrella basket shape. FIG. 14B is a cross-sectional view of a device showing the substantially rigid center rod 59 and endcap 57 as a one-piece assembly, which constitutes the inner most core and endcap of the device.

FIG. 15 shows a cross-sectional schematic where the double-umbrella basket 48 with its inner membrane covering 50 (also referred to herein as a membranous covering) fully expanded to create the desired shape at surgical site 150 and also pressing and depositing the particles 172 onto the surface of the bone wall cavity 154, according to one embodiment of the invention.

FIG. 16 shows when the double-umbrella basket 48 is deployed and the struts 51 are curved outward; the particles 172 contained within the inner membrane covering 50 may be released when the outer membrane covering 53 (also referred to herein as a membranous covering) ruptures, according to one embodiment of the invention. The left-hand drawing is a side-sectional view, while the right-hand drawing is an end view through line A-A of the left-hand drawing.

FIG. 17 is a cross-section that shows the cannula introducer 47 and the double-umbrella basket 48 with any remaining outer membrane covering 53 removed from the surgical site 150 and the particles 172 deposited on the wall of the bone wall cavity 154, according to one embodiment of the invention.

FIG. 18 is a cross-section that shows the surgical site 150 in bone 140 filled with a mixture of cement precursor 177 containing particles 172 and particles 174, according to one embodiment of the invention.

FIG. 19 is a cross-section that shows the cement precursor 177 in bone 140 having hardened to form cement 176 containing particles 172 and particles 174, according to one embodiment of the invention.

FIG. 20 is a cross-sectional schematic illustrating an introducer tool 208 within the cancellous bone 206 of a vertebral body 200 according to one embodiment of the invention.

FIG. 21A illustrates a schematic of a cavity forming tool 220, according to one embodiment of the invention.

FIG. 21B illustrates a schematic of a particulate-dispensing tool 234, according to one embodiment of the invention.

FIG. 22 illustrates the cavity forming tool 220 shown in FIG. 21A coaxially advanced through the central lumen 216 on access cannula introducer 208, according to one embodiment of the invention.

FIG. 23 is a cross-sectional schematic illustrating the cavity forming element 228 of the cavity forming tool 220 having been transformed to its enlarged profile, according to one embodiment of the invention.

FIG. 24 is a cross-sectional schematic illustrating the access cannula introducer 208 after removal of the cavity forming tool 220, according to one embodiment of the invention.

FIG. 25 is a cross-sectional schematic illustrating the particulate-dispensing tool 234 being coaxially advanced through the central lumen 216 of access cannula introducer 208, according to one embodiment of the invention.

FIG. 26 shows the access cannula introducer 208 after withdrawal of the particulate dispensing tool 234, according to one embodiment of the invention.

FIG. 27 is a cross-sectional schematic illustrating the vertebrae 200 after removal of the access cannula introducer 208 and introduction of implant 252, according to one embodiment of the invention.

FIGS. 28A-E schematically illustrate steps of creating a cavity within a bone utilizing an expandable device with generally rectangular-shaped struts, according to some embodiments of the invention.

FIG. 29 schematically illustrates depositing particles within a bony cavity utilizing an expandable device with generally rectangular-shaped struts, according to some embodiments of the invention.

FIGS. 30A-B schematically depicts a method of spreading particles within a bony cavity utilizing an expandable device with generally rectangular-shaped struts, according to some embodiments of the invention.

FIGS. 31A-E schematically illustrate steps of creating a cavity within a bone utilizing an expandable device with generally “bow-tie” shaped struts, according to some embodiments of the invention.

FIG. 32 schematically illustrates depositing particles within a bony cavity utilizing an expandable device with generally “reversed bow-tie” shaped struts, according to some embodiments of the invention.

FIGS. 33A-B schematically depict a method of spreading particles within a bony cavity utilizing an expandable device with generally “reversed bow tie” shaped struts, according to some embodiments of the invention.

### DETAILED DESCRIPTION OF THE PREFERRED EMBODIMENT

The composition regime of a composite exemplified by an optimal weight fraction of particles (which is greater than about 25%, and less than about 35% in a preferred embodiment) in the cement can be described as a crack-arresting regime. Based on the known proportion of the particles and the polymer, it can be expected that such a composite contains a substantially continuous phase of hardened acrylic cement that surrounds particles, which infrequently touch each other. It is believed that cracks, which originate in the substantially continuous polymeric phase, are only able to propagate for a short distance before they reach the hole of a particle, which then arrests the growth of the crack propagation. If a composite contains an even higher volume fraction of particles, it can exhibit another regime of behavior in vivo. In such a situation, there would again be at least some of a continuously interconnected phase of hardened acrylic cement, but, at the same time, many of the particles would have direct contact with one or more adjacent particles. If the particles are bioresorbable, resorption of the particles and ingrowth of new bone may occur simultaneously and could be expected to eventually leave ingrowth of natural bone into the bone cement. The situation in which bone has grown about 2 mm or more into the polymeric phase can be expected to yield especially good interfacial shear strength. This situation can be referred to as the “bone ingrowth” regime.

The present invention provides an improved bone cement composite, whose bulk provides the fatigue life typically attainable with particle-containing cement, and also which further exhibits a greater interfacial shear strength at the bone-cement interface than would normally be obtained using pure bone cement.

The bone cement composite of the present invention is believed to operate in the crack arresting regime throughout most of its bulk and in the bone ingrowth regime near the interface with natural bone. The enhanced shear strength at the bone-cement interface may allow many more implants to last the lifetime of the patient without ever needing revision surgery.

In general, the foregoing is achieved in accordance with the present invention by providing a bone cement composite in which the local volume fraction of particles in the composite is spatially non-uniform in a controlled manner.

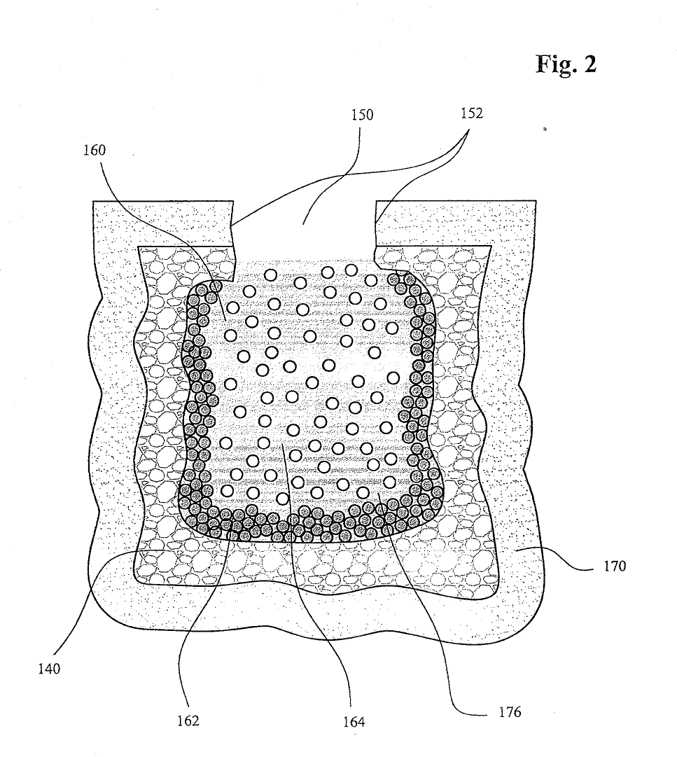

One embodiment of the invention is the configuration of the composite as it exists in the body of a patient after completion of the surgical procedure, in which the composite has two regions. This is illustrated in FIG. 2. FIG. 2 illustrates a portion of a bone 140, which has a surgical site 150. The surgical site 150 may have a potential cavity opening 152, and the potential cavity may contain composite 160. Composite 160 may comprise first region 162 and second region 164, which differ from each other in some respect. In FIG. 2, region 162 generally adjoins the bone 140, which defines the boundary of potential cavity opening 152. In some embodiments, region 164 may be generally surrounded by region 162 and generally may be free from contact with bone 140 which forms the boundary of potential cavity opening 152 In some embodiments, localized exceptions or anomalies can be present as well.

The invention will be described primarily herein in the context of introducing the composite bone cement described herein into a vertebral body. However, it is contemplated that the composites disclosed herein can be introduced into a wide variety of bones throughout the body, and optionally in conjunction with the prior or concurrent formation of a cavity. Such bones may include, for example, the pelvis, the femur, the fibula, the tibia, humerus, ulna, radius, ribs, or various component structures of the cranial or facial skull. A wide variety of applications for this method would be appreciated by one of ordinary skill in the art, and can include therapeutic intervention for degenerative, infiltrative, traumatic and/or malignant defects of bone that include but are not limited to: Paget's disease, osteoporosis, osteomalacia, myeloma, metastatic epithelial malignancies, primary or metastatic sarcomas, osteogenesis imperfecta, osteochondromas and/or other non-metastatic deformative defects of bone including hemangiomas.

In addition, the invention can be described in the context of introduction of the composite into a vertebral body to restore vertebral body height, or minimize further degeneration of the vertebral body. In addition to filling a cavity in a bone, the composite of the present invention may be utilized in any of a variety of other applications in which adhesion of a bone or non-bone prosthesis or device to a bone is desirable. For example, the composite of the present invention may be utilized to assist in the fixation of any of a variety of devices to an interior or exterior surface of a bone, such as, for example, fixation of a medullary nail or rod, screws, plates, and other stabilization, fixation or mobility preservation hardware. Specific applications can include fixation of a total shoulder or total hip replacement, such as by fixation of a prosthesis stem within a medullary canal. The composite can also be used for reconstructive applications, e.g., reconstruction of congenital abnormalities, posttraumatic reconstruction of facial structures, pelvic and/or other bony sites, or postresection reconstruction in patients with epithelial or bony malignancies, including, but not limited to, head and neck carcinomas, pelvic sarcomas or discrete bone metastases following resection or other ablative procedures including radiofrequency (RF) and high intensity focused ultrasound (HIFU) ablation therapies.

The composite of the present invention can additionally be utilized to assist in the attachment of any of a variety of bone anchors, suspension slings, or implantable diagnostic or therapeutic devices to bone, as will be apparent to those skilled in the art in view of the disclosure herein. Further, the composite of the present invention may additionally be utilized to stabilize or secure a bone graft, allograft, synthetic bone grafts, or other implants within or adjacent a bone.

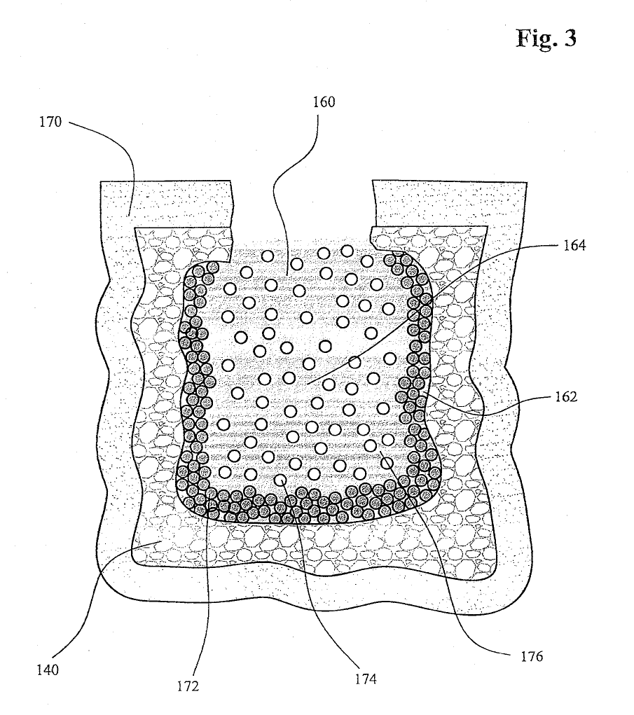

Regions 162 and 164 are illustrated in more detail in FIG. 3 by further showing that regions 162 and 164 may further contain, respectively, particles 172 and 174. In FIG. 3, as well as in other similar figures herein, the particles 172 and 174 are illustrated as being spheres of equal diameter. However, it is to be understood that this is only an idealization for ease of illustration, and in reality any of the particles 172, 174 could vary in any one or more of the following attributes, such as, for example, size, shape, size distribution, shape distribution, or other geometric characteristics. Particles 172 and 174 could be either identical to each other or different from each other in some respect, as discussed elsewhere herein.

In describing the presence of particles in cement, the term concentration (of particles) is used herein as a generic term referring to either volume fraction or weight fraction of particles in the cement. If a concentration of particles is reported as a weight fraction, as would be understood by one of ordinary skill in the art, a corresponding volume fraction of particles can be calculated if the mass densities of the particle material and the mass density of the cement are known. If the mass density of the particle material and the mass density of the cement happen to be identical, then the weight fraction and the volume fraction of the particles would be numerically identical. If the two mass densities are unequal, then numerical calculations can convert from mass fraction to volume fraction or vice versa, as known by those with skill in the art.

In some embodiments, at least some of the composite 160 can contain a continuous phase of cement 176, which may have dispersed solid particles 172 and 174 within the cement. In both region 162 and region 164, the composite could have a non-zero local volume fraction of the particles 172 and 174. The non-zero local volume fraction of particles 172 and 174 may be such that the composite has fatigue life which is longer than the fatigue life of particle-free or substantially particle-free cement. Within the composite, the local concentration of the particles 172 in region 162 may be different from the local concentration of the particles 174 in region 164. As illustrated in FIGS. 2 and 3, region 162, adjoining bone, could have a greater non-zero local volume fraction of particles 172, and region 164, generally not adjoining bone, may have a lesser non-zero local volume fraction of particles 174.

In the region designated 164, away from the immediate vicinity of the bone-composite interface, the concentration of particles 174 may be described by a weight fraction designated α. For example, this concentration of particles 174 may be in the range of approximately at least about 10% by weight to approximately no more than about 50% by weight. In another embodiment, the concentration of the particles 174 in region 164 may be in the range of approximately at least about 20% by weight to approximately no more than about 40% by weight. In yet another embodiment, the concentration of particles 174 in region 164 are preferably at least about 25% by weight but no more than about 35% by weight. In another embodiment, the concentration of particles 174 in region 164 is preferably about 30% by weight. The concentration of the particles 174 may be selected, at least in part, so as to provide desired fatigue properties of the resulting composite. Although about 30% weight concentration of particles has been reported in the literature to be the optimum concentration for the reported combination of materials, more generally, the particle concentration which produces the best fatigue properties may be unique to particular combinations of particle composition and properties and cement composition and properties. In region 164 of the composite, the properties of the composite may be such that the weight-bearing behavior can be described as being in the “crack arresting” regime. In this regime, generally speaking, most of the particles 174 may be immediately surrounded by cement 176, without being in direct contact with other particles 174. In FIG. 2, region 164 is illustrated as containing particles 174, in which at least most of the particles 174 do not touch any other particle 174. On average, such particles 174 may be separated from each other by only a small number of particle diameters or even by just a fraction of a particle diameter. This situation means that, on average, such a distance is the greatest length to which a crack in cement 176 is likely to grow before encountering a particle 174 which would arrest the growth of the crack propagation. Once the crack is arrested, additional cyclic loading may be needed to either initiate new crack(s) or propagate existing crack(s). This is believed to be the primary mechanism by which the presence of particles such as particles 174 can improve fatigue properties in this regime. However, other mechanisms may contribute to enhance the fatigue life in this regime as well.

In FIG. 2, the more densely packed region 162 is illustrated as containing particles 172, in which most of particles 172 directly touch other particles 172. At the same time, particles 172 may be at least partially surrounded by cement 176. In the immediate vicinity of the bone-cement interface, in region 162, the cement composite may have a local volume fraction of particles 172 which is designated by β. It can be noted that, based on geometric packing considerations and with assumption of spherical equally-sized particles, the maximum possible volume fraction of particles under any circumstance is no more than about 70%, with some variation possible depending on exact packing arrangement of particles and with the possibility that if there are multiple sizes of spherical particles or if there are non-spherical particle shapes, the number could be somewhat higher than about 70%. As discussed elsewhere herein, with knowledge of the respective mass densities of the particles and the cement, a relationship could be calculated by one of skill in the art between local volume fraction of particles and the local mass fraction of particles. In region 162, which is in the immediate vicinity of the bone-cement interface, the concentration of particles 172 may be in the range of about 50% to about 80% by weight, or more preferably about 60% to about 80% by weight. In such an embodiment, a significant fraction, such as more than about 50% of the particles 172 in region 162 of the composite, may have direct contact with a nearby particle 172. In other words, a particle 172 which is directly in contact with bone, could biodegrade, and replaced by new bone. Then, the bone can further come in contact with another particle 172 which had been in contact with the earlier-existing particle 172 before that particle was replaced by bone. Upon this occurrence, there may be a repetition of the method of particle resorption and bone ingrowth. By this method, ingrowth of a continuously connected network of natural bone into the composite may proceed for a distance of some number of particle diameters into the composite. For this reason, such a composite may be referred to as being in the “bone ingrowth regime.”

In general, in the present invention, in the immediate vicinity of the bone-cement interface (region 162), the composite may have a local volume fraction of particles 172 that is larger than the local volume fraction of particles 174 away from the immediate vicinity of the bone-cement interface (region 164). Region 162 may have a local volume fraction of particles that touch others, which puts it in the bone ingrowth regime. This is believed to help improve the strength of the bone-cement bond, such as the interfacial strength in shear. This is because shear strength is provided by bone ingrowth, and the amount of the ingrowth can be expected to increase with the concentration or volume fraction of the particles and the degree to which the particles contact each other to form inter-touching particles (which can be expected to increase with the local concentration or volume fraction of the particles). A region of composite having a relatively high local concentration of particles can be expected to contain a substantial number of particles 172, which directly touch other particles 172. The presence of particles 172, which directly touch other particles 172, can be expected to create interconnected particles, which in turn can be expected to help produce bone ingrowth by bone resorption and ingrowth. Again, however, it is not wished to be restricted to any of these theories or explanations.

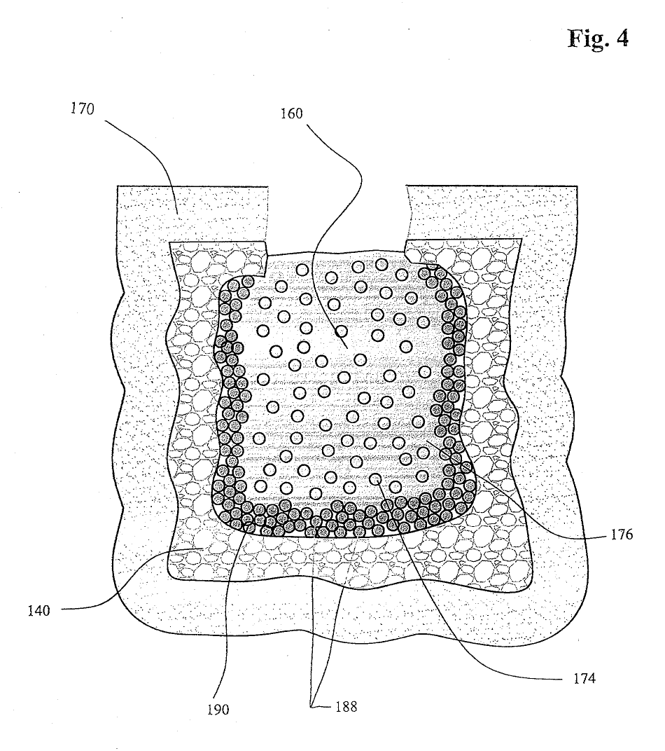

The immediate vicinity of the bone-composite interface can be defined herein to mean a distance of somewhere in the range of approximately 0.1 mm to approximately 2 mm, or no more than about 2 mm. Also, a local particle concentration can be defined as the weight or volume (depending on whether volume fraction or weight fraction is being discussed) of particles contained in a space, divided by the total weight or volume of all material contained in that space, wherein the space is at least approximately equiaxial in all three orthogonal dimensions and has a volume which is sufficient to contain at least approximately 3 particles or fractions of particles. For present applications, a typical average overall dimension or diameter of the particles 162 and 172 may be at least about 50 micrometers to no more than about 500 micrometers in some embodiments, and at least about 150 micrometers to no more than about 300 micrometers in another embodiment. In FIG. 3, the particles 174 are illustrated as being completely surrounded by cement. However, this is not essential and another embodiment of the invention can include particles 174 which are less than completely surrounded by cement. In FIG. 4, particles 188 are in only partial contact with cement. Furthermore, there may be particles such as particles 190, which are not in contact with any cement.

It is further possible, in still another embodiment of the invention, that even though most of the bone-composite interface occurs with the bone 140 contacting region 162 as illustrated in FIG. 2, there might be some isolated places where such an identifiably different region 162 does not separate region 164 from cancellous bone 140, and, for example, the region 164, operating in the crack-arresting regime, might contact cancellous bone 140. The outer layer of cortical bone 170 is also shown.

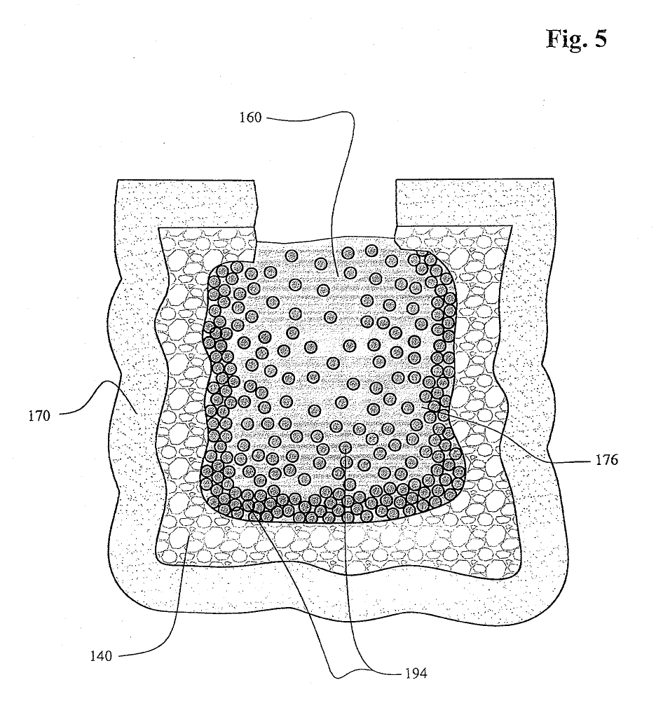

Description, for example of FIGS. 2, 3 and 4 herein refers to a composite which contains identifiable regions such as regions 162 and 164 within the composite. Alternatively, as yet another embodiment of the invention, it is possible that the local volume fraction of particles may exhibit spatial non-uniformity, but without always having sharply-defined identifiable regions 162 and 164 as already illustrated. For example, there may be a gradient of local volume fraction of particles from one place to another within the composite. This is illustrated in FIG. 5. In FIG. 5, the particles 194 closest to the bone generally touch other particles, and the particles 194 in the interior of the composite generally do not directly touch other particles, but the variation between these two situations is more gradual than was illustrated in FIGS. 3 and 4. In FIG. 5, particles 194 generally represent the same particles as particles 172 and 174 in FIGS. 3 and 4, but in FIG. 5, the local volume fraction of particles 194 varies spatially in a somewhat continuous variation, rather than in an approximately stepwise manner. A still further possibility is that there could be identifiable regions such as regions 162 and 164, such that within an individual region the concentration of particles is substantially constant, but in the immediate vicinity of where the two regions meet each other, there could be a gradient of particle concentration.

In any situation (gradient or identifiable regions or other situations), the distribution of local volume fraction of particles can be such that the local volume fraction of particles within the cement may be greater in the immediate vicinity of the bone interface than it is away from the bone interface. In general, the local particle concentration may be spatially non-uniform, and may be non-zero substantially everywhere throughout the composite. These spatial variations of particle concentration may be controlled variations which achieve desired particle concentrations in specific places. The desired particle concentrations may be chosen for reasons related to biological considerations or fracture mechanics, as described elsewhere herein.

In other embodiments, it is possible that some localized region of zero local particle concentration may exist, while, at the same time, there exists a spatially non-uniform distribution of local particle concentration in that portion of the composite which does contain particles. For example, this may occur in connection with the filling of cavities in smaller bones such as vertebrae as compared to long bones in total knee and hip joint replacements.

Materials

The particles may be biocompatible and/or bioresorbable. Specifically, in an embodiment which contains identifiable regions such as regions 162 and 164, at least the particles 172 in region 162 (which adjoins natural bone 140) may be bioresorbable. More generally, such as in embodiments that have a gradient, at least the particles, which are in the immediate vicinity of the interface with natural bone, may be bioresorbable. In a region in which bone ingrowth is desired, such as region 162, the bioresorbability of the particles 172 in that region, may allow those particles to be replaced by natural bone for the formation of a strong interfacial bond. More interiorly in the composite, such as in region 164, the particles may also be bioresorbable, but are not required to be.

Any of the particles 172 and 174 may include one or more of the following materials: inorganic bone; demineralized bone; natural bone; bone morphogenic protein; collagen; gelatin; polysaccharides; polycaprolactone (PCL); polyglycolide (PGA); polylactide (PLA); DLPLG which is a copolymer of PLA and PGA; polyparadioxanone (PPDO); other aliphatic polyesters; polyphosphoester; polyphosphazenes; polyanhydrides; polyhydroxybutyrate; polyaryetherketone; polyurethanes; magnesium ammonium phosphate; strontium-containing hydroxyapatite; beta tricalcium phosphate; other forms of calcium phosphate. The particles could contain carbon in any form appropriate for use within the human body. The particles may be either osteoconductive, osteoinductive or both. If the particles are at least osteoconductive, they have been shown by Y. K. Liu, J. B. Park, G. O. Njus, and D. Stienstra, “Bone-particle-impregnated bone cement: An in vitro study,” Journal of Biomedical Materials Research, Vol. 21, 247-261, 1987 that those inter-touching particles would promote the formation and ingrowth of bone into the cement through simultaneous osteoclastic and osteoblastic activities. If the particles are osteoinductive and the exothermic excursion of cement such as PMMA were to destroy some or all of the osteoinductive properties of the osteoinductive material, then its osteoconductivity would still remain.

As will be appreciated by one of ordinary skill in the art, examples of osteoconductive particle types include inorganic bone particles, collagen, beta tricalcium phosphate and other forms of calcium phosphate. Examples of osteoinductive particles include osteogenic protein-1, demineralized bone matrix (DBM) and bone morphogenic protein-2. Examples of both osteoconductive and osteoinductive particles include natural bone, e.g., allogenic and autogenous bone grafts as well as collagen mineral composite grafts, e.g., collagen in combination with hydroxyapatite and tricalcium phosphate.

The particles 172 and 174, or the particles in any individual region, may be a mixture of more than one kind of particle, and may have a distribution of sizes, shapes and other properties. The particles could be of any shape. In some embodiments, the particles could even have a shape which is as elongated or non-equiaxial as a fiber. Fibers can be advantageously useful as strengthening agents in composite materials.

The particles 172 in region 162 and the particles 174 in region 164 could be substantially identical to each other in all their physical properties such as composition and geometric and dimensional properties. Alternatively, the particles 172 and 174 in the two regions 162 and 164 could differ from each other in any one or more or any combination of the following properties: composition, biocompatibility, resorbability or resorption rate, size, shape, size distribution, shape distribution, or any other property. In any individual region, the composition, size shape, and any other properties of the particles in that region may be chosen appropriately to produce a composite having mechanical and material or other properties which are desired for that individual region.

In the situation where there is a gradient of particle concentration, there could also be differences from one place to another place in any of the physical properties of the particles, as just mentioned.

The particle size or distribution of particle sizes can be varied widely, depending upon the composition of the particles and the intended clinical performance. In general, particles having a size, for example, of at least about 150 microns to no greater than about 300 microns, can be optimal for osteoconductive ingrowth of bone to the composite (see J. J. Klawitter and S. F. Hulbert “Application of Porous Ceramics for the Attachment of Load Bearing Internal Orthopedic Applications,” J. Biomed. Mater. Res. Symp., 2(1), 161-229, 1972), and; J. B. Park and R. S. Lakes “Biomaterials: An Introduction—Second Edition,” Plenum Press, 1992, pp 177-178.).

In the present invention, the bone cement may be non-resorbable or may have only a very slow rate of absorption such as taking more than about 50 years to resorb in the environment of the human or animal body. The bone cement may include polymethylmethacrylate (PMMA) cement. Alternatively, or in addition, the bone cement could include any one or more of: hydroxyethyl methacrylate (HEMA); polyalkanoate; polyetherurethane; polycarbonate urethane; polysiloxaneurethane; and polyfluoroethylene. Agents that may be included in the composition of the PMMA/particulate aggregate may include thrombin, fibrinogen, epsilon-aminocaproic acid (Amicar) or other agents to prompt local clotting at the perimeter of the cavity; particulate or soluble antibiotics to preclude infection at the procedure site; growth factors to stimulate either neovascularization or otherwise facilitate incorporation of the high concentration particulate component of the implanted material, including but not limited to endothelial growth factors such as VEGF; G-CSF, GM-CSF, or thrombopoietin; contrast material to enhance visualization of the implanted material during and after the procedure; in the case of malignant replacement or bone destruction, chemotherapeutic agents in either a soluble, gel or solid phase may be introduced including but not limited to adriamycin and cisplatin; single or multiple osteogenesis-enhancing agents may also be incorporated into the compound before, during or after introduction of the cement and bioresorbable particles.

Methods

Introduction of the composites of the present invention into a bone, either alone or in combination with other implants, can be accomplished in any of a variety of ways as will be appreciated by those of skill in the art. In general, in the example of filling a cavity formed in a bone, the particle gradient can be accomplished by introducing a layer of particulate in contact with the surface of the bone which defines the cavity, and thereafter introducing bone cement to sandwich the particulate layer between the bone cement and the bone surface to which adhesion is desired. The wall of the cavity may be completely covered with a layer of particulate, or only selected portions may be covered with particulate, depending upon the desired result.

Methods for creating or modifying a cavity, whether de novo or preexisting include, but are not necessarily limited to: percutaneous or open aspiration, surgical resection, radiofrequency (RF) ablation or high intensity focused ultrasound (HIFU) ablation, thermal ablation, mechanical displacement, enzymatic or other solubilizing processes, or introduction of incompressible fluid by a variety of mechanical means known to those skilled in the art.

In some embodiments, the particulate can be introduced into the cavity in dry powder form, such as by spraying or extruding the powder under pressure from a deployment lumen or spray head. Alternatively, the dry powder may be advanced into contact with the walls of the cavity such as by carrying on the surface of an expandable member such as a balloon, sponge, or other expandable structure.

In other embodiments, the particulate can be introduced together with one or more carriers. For example, the particulate can be introduced in the form of a suspension or slurry, such as an aqueous slurry, which may additionally include viscosity enhancing agents. Some agents, as known in the art, can also be included either to modify viscosity or to modify other characteristics of the cavity and the procedure, including thrombin, fibrinogen or other thrombogenic agents; enzymatic substances such as hyaluronidase or other lytic substances; osteogenesis-enhancing agents; and antimicrobial agents such as antibiotics.

In one implementation of the invention, the particulate is carried in a paste or gel having sufficient viscosity and adhesion characteristics that it will adhere in a thin layer to the bone surface. A paste or suspension of the particulate may be distributed across the surface of the bone which defines the cavity using any of a variety of devices, such as spreaders, sprayers, or a balloon or other expandable structure to distribute the particulate across the surface of the bone.

A variety of surgical approaches to access a vertebral body have been employed and described. Selection of an approach is dependent upon clinical judgment, the particular defect addressed, and the vertebral level, as some levels are more amenable to an anterior rather than posterior approach. Options may, however, include lateral approaches; transpedicular access; a variety of direct or oblique approaches generally described as ‘posterior’ approaches can be employed or envisioned by those skilled in the art.

Depending upon the nature of the carrier, the layer of particulate in contact with the surface of bone may be permitted to harden or partially harden prior to introduction of the remainder of the bone cement to form the core of the formed in situ implant.

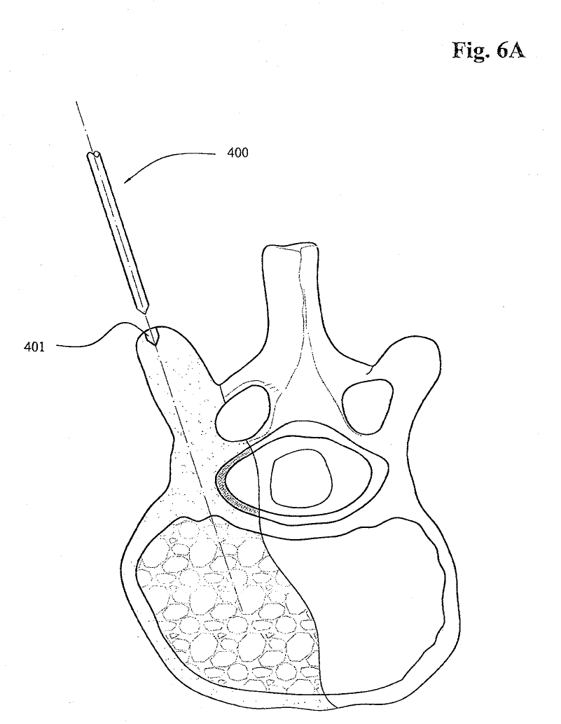

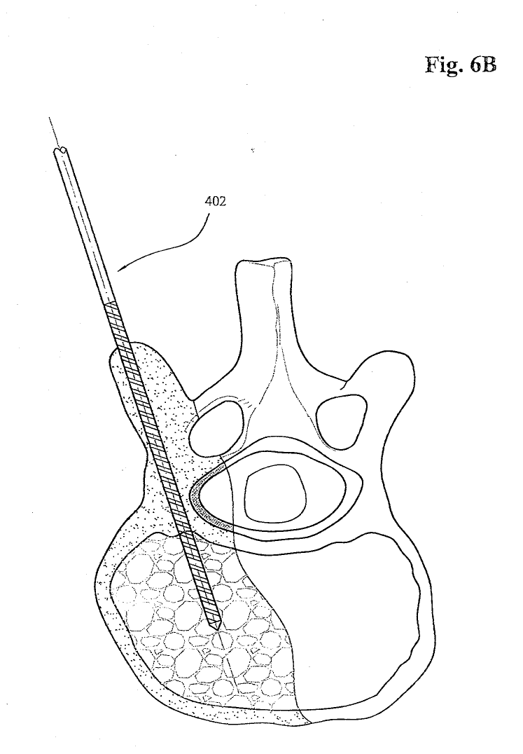

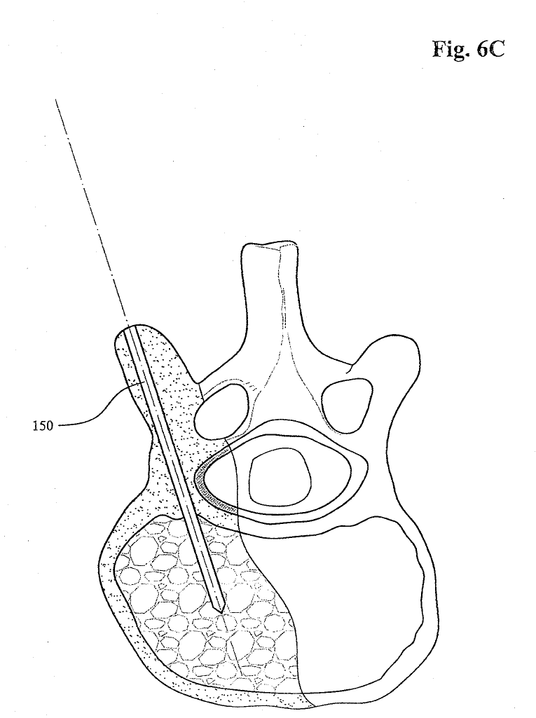

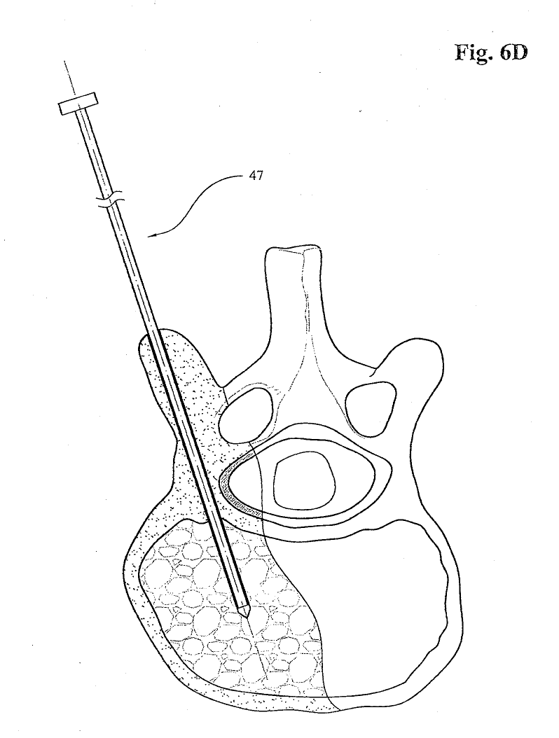

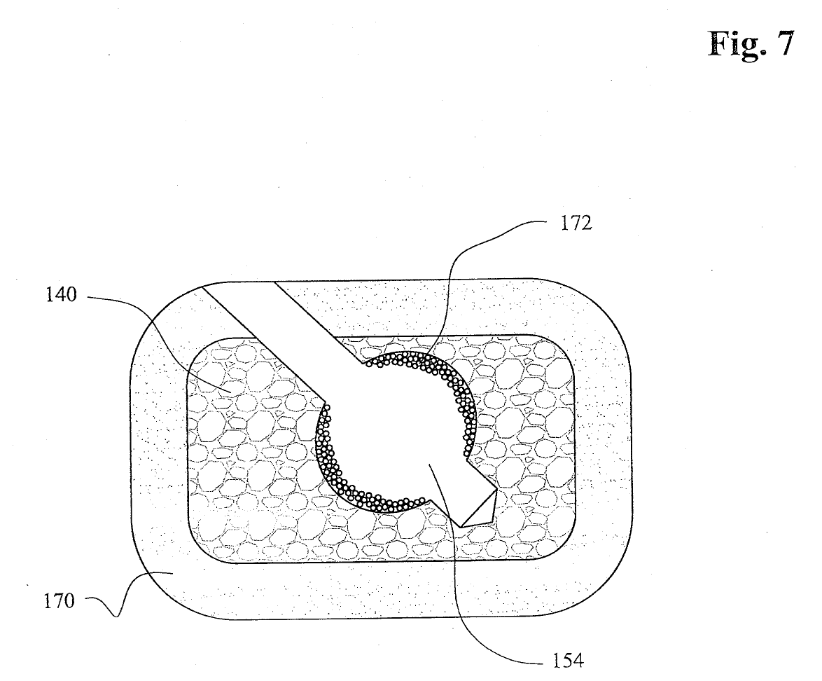

Another aspect of the invention includes a method of depositing the described particles to form the differential composite. Initially, as illustrated in FIGS. 6A-6D, a surgical site 150 may be created in a bone 140, using a bone drill or other tools and procedures known by those skilled in the art. One method of depositing the layer of particles 172 is through a cannula introducer 47 that deploys a mechanically expandable support, as described elsewhere herein. Then, as illustrated in FIG. 7, a layer of particles 172 may be deposited on at least some of the interior wall of the cavity 154 in bone 140. The thickness of the layer of particles 172 can vary, in some embodiments, from at least about 0.1 mm to no more than about 4 mm. It is also possible to deposit the particles 172 by a simpler method such as by using hand-held tools such as a spatula, swab, or other applicator.

Particles in dry powder form may alternatively be introduced using compressed air, such as from a syringe, squeeze bulb, or other source. The interior surface of natural bone, especially a freshly-compressed or crushed interior surface of natural cancellous bone, may be moist or even wet with blood, bone marrow, and interstitial fluid. This moisture or wetness may help to hold particles 172 in place during and shortly after this application procedure. As still another alternative, the particles 172 may be deposited in the form of a paste, possibly made by mixing the particles together with water, blood, other bodily fluids, or other carriers having appropriate physical consistency.

At an appropriate time, a cement precursor 177 may be prepared by the doughing method as discussed elsewhere herein, such that cement precursor 177 will harden with the passage of a period of time, such as a few minutes, to form cement 176. The cement precursor 177 as prepared may be freshly-mixed polymeric cement precursor having a known hardening time which is appropriate for the surgical procedure, such as less than about 5 minutes. For example, the cement precursor may contain appropriate hardeners or accelerators or radio-opaque additives as are known by those skilled in the art. Cement precursor 177 may contain particles 174, with a particle density represented by α. This mixture may be introduced into the cavity 154, which is created during deposition of particles 172, and may be pressed against the layer of particles 172, which adjoins the wall of cavity 154 in bone 140.

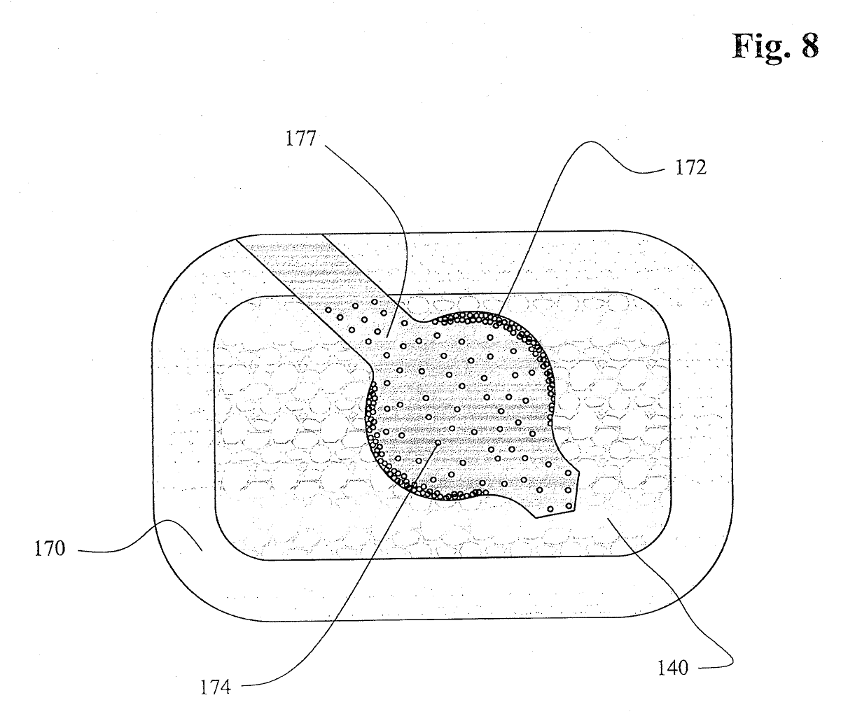

FIG. 8 shows, schematically, the appearance of the surgical site immediately after this filling has been performed. This cement precursor 177 may contain particles having a particle concentration represented by α, which is equal to or approximately equal to the desired final concentration of particles 174 in cement 176 in the bulk interior region 164. As discussed elsewhere herein, the particles 174 contained in the cement precursor 177 may be substantially identical to the previously-introduced particles 172 or, alternatively, the particles 174 contained in the cement precursor 177 may be different in some characteristics from particles 172. Cement precursor 177 containing particles 174 may be introduced into the cavity 154 with sufficient pressure to approximately conform to the shape or size of the surgical site 150. The injection pressure may be limited by the egress of bone cement from the cortical bone as can be detected by fluoroscopy.

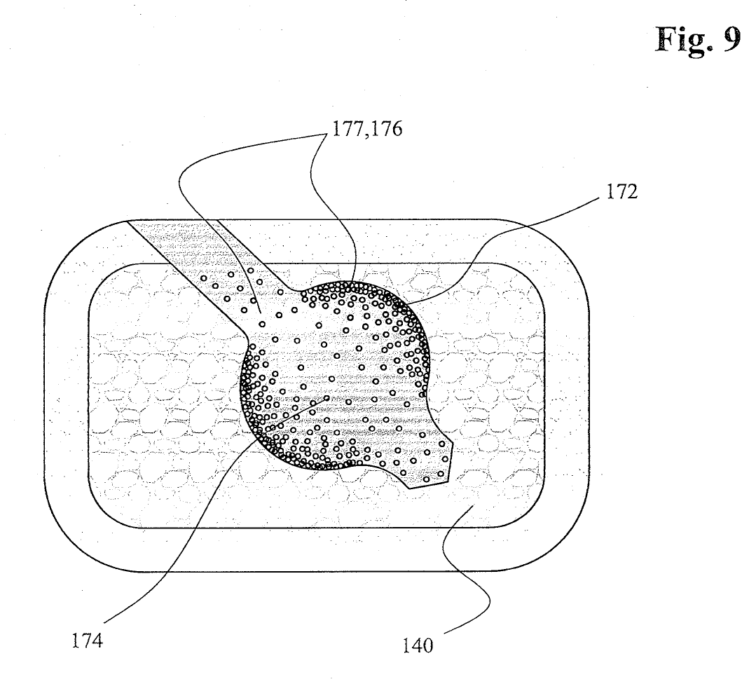

During the time before the cement precursor 177 hardens, and depending upon the pressure with which the cement precursor 177 containing particles 174 is introduced into cavity 154, some of the cement precursor 177, a mixture of PMMA and particles 174, may move into the interstices between the previously-introduced particles 172. This movement may be caused or aided by application of pressure to cement precursor 177 containing particles 174. Additionally, this movement may bring about the situation shown in FIG. 9, wherein the cement precursor 177 substantially surrounds both the originally-introduced particles 172 and the particles 174, which were mixed in with the cement precursor 177. The desired volume fraction of particles 172 of the region 162 can be achieved in part by controlling the amount of the particles 172 deposited on the wall of the cavity 154 in bone 140. With the passage of time, cement precursor 177 may harden and become cement 176. This may help to bring about the formation of a bone-cement interface region 162, having a relatively large local particle concentration β. The particle concentration β in region 162 may be chosen so as to put that region into the optimal bone-ingrowth regime to achieve the maximum interfacial shear strength, while the bulk of the bone cement (region 164) will have the longest fatigue life since it may have a local particle concentration α, which is optimized for the arrest of crack propagation.

It should also be noted in FIG. 9 that the illustration of a distinct boundary between the bone-contacting region 162 and the bulk interior region 164 is partly for convenience of elucidation. It is possible that the local volume fraction of particles in the composite may change somewhat gradually from the local volume fraction of particles (which is characteristic of the bone-contacting region 162) to the local volume fraction of particles, which is characteristic of the bulk region 164. Certain techniques, which may be used to achieve differential particle density, are discussed elsewhere herein.

Injecting cement precursor 177 (containing particles 174) or in general injecting any fluid into an internal cavity in bone that may or may not involve a deployable double-umbrella basket, as described elsewhere herein, may be done using sufficient pressure well known to those skilled in the surgical procedure. Such pressure may be limited in magnitude to avoid causing any catastrophic failure of the bone involved in the surgical procedure. If desired, such pressure may be maintained for a sufficiently long period of time so that cement precursor 177 remains under pressure until it completes at least a substantial portion of its transformation into cement 176. Following the deposition of all of the described substances and upon (if necessary) allowing an appropriate amount of time for the cement precursor 177 to harden to form cement 176, the surgical site may be closed using well-known surgical techniques.

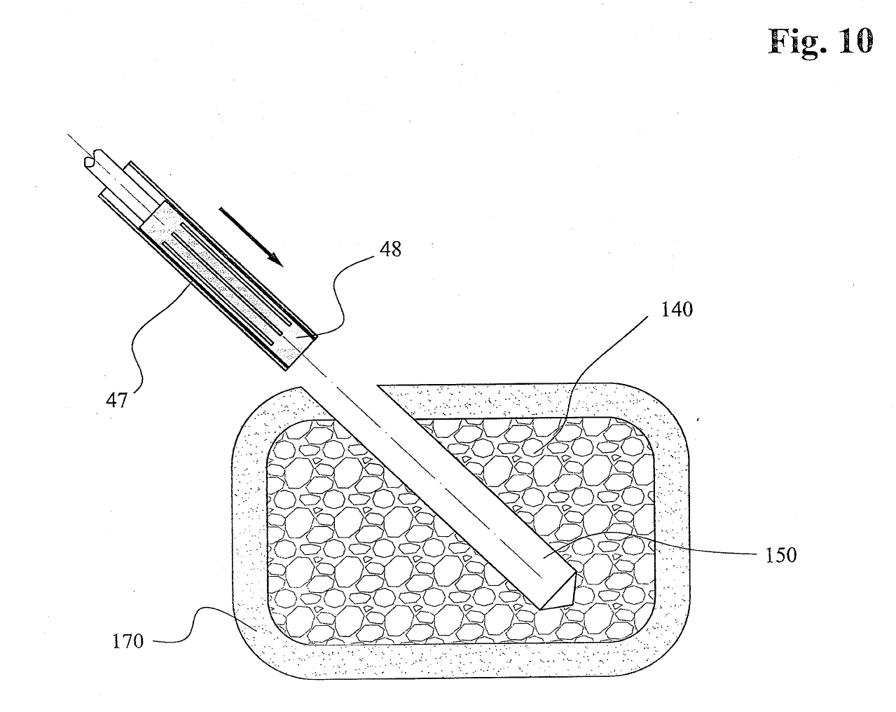

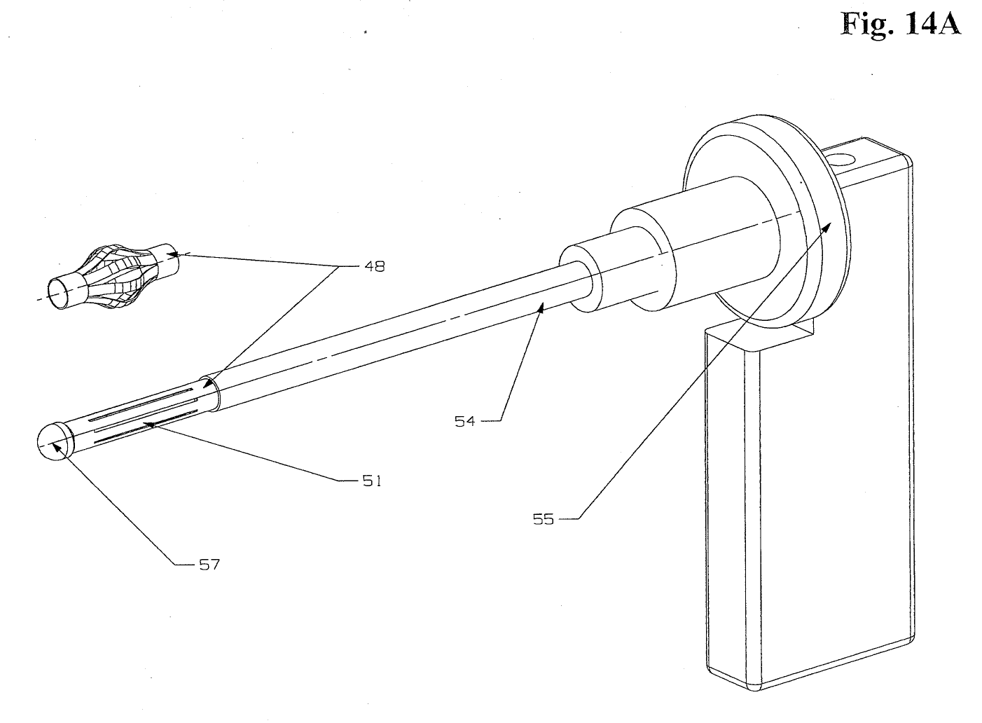

As part of the described procedure, in some embodiments, it may be possible to deposit the layer of particles 172 by using a cannula introducer 47 and a media deployment device, such as a deployable mechanically expandable basket device. One embodiment of this procedure is illustrated further in FIGS. 10-19; another embodiment of the associated device is further illustrated in FIGS. 14A-B.

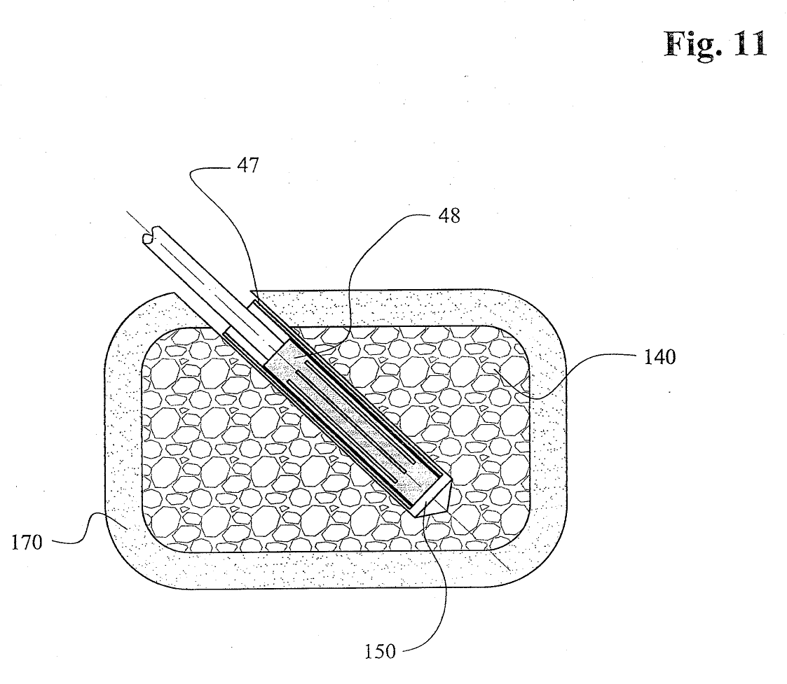

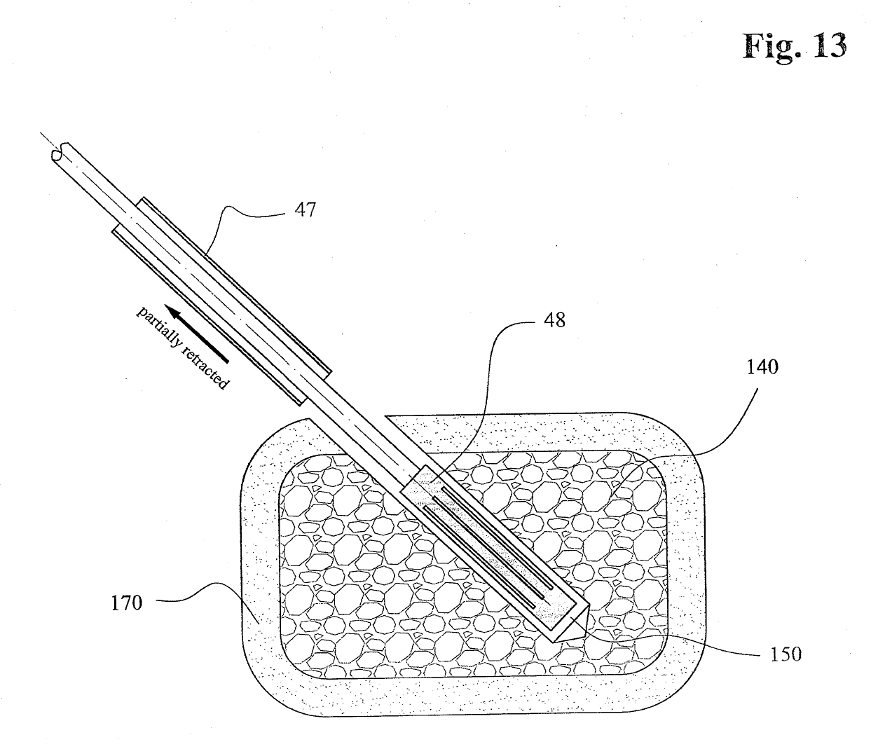

As shown in FIG. 11, the apparatus can include a tubular introducer 47 through which a cavity forming tool such as an expandable basket 48 may be deployed. In some embodiments, the introducer has a diameter of between about 4-8 mm, more preferably between about 5-6 mm. The length of the introducer is generally within the range of from about 12 cm to about 30 cm in some embodiments. In FIG. 11, the introducer 47 and double-umbrella basket 48 assemblies may be placed within a bone 140 using surgical techniques known to those skilled in the art. As shown in FIG. 13, the introducer 47 then may be partially retracted and the double-umbrella basket 48 may be positioned for deployment.

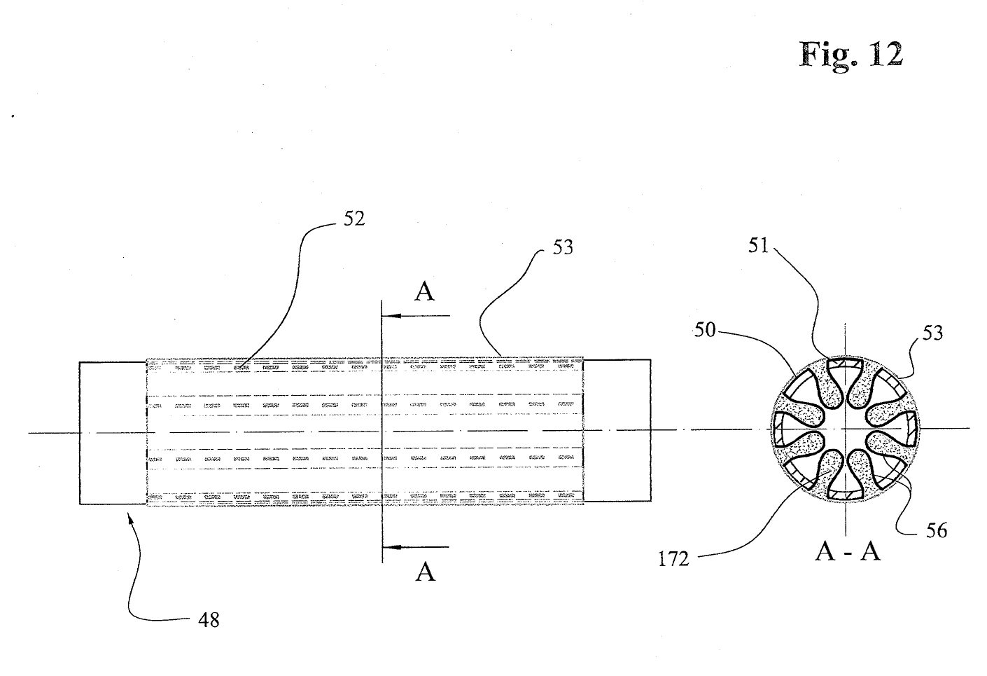

As shown in FIG. 12, the expandable basket 48 may comprise a plurality of flexible struts 51 which may be approximately oriented in the same length-wise direction to each other. In an undeployed state, the struts 51 generally have an axial configuration. The struts 51 may be capable of assuming a deployed state in which the struts 51 are curved outward, or radially expand, thus creating an end-to-end double-umbrella basket shape in some embodiments. In between each strut 51 of the double-umbrella basket 48 is a continuous inner membrane covering 50 that holds particles 172 within the undeployed double-umbrella basket 48. The particles 172 are further contained by a biocompatible and bioresorbable outer membrane covering 53, which may be firmly affixed to struts 51. The outer membrane coverings 53 between struts 51 are preferably prefabricated with one or more severable regions, such as perforated tear slots 52.

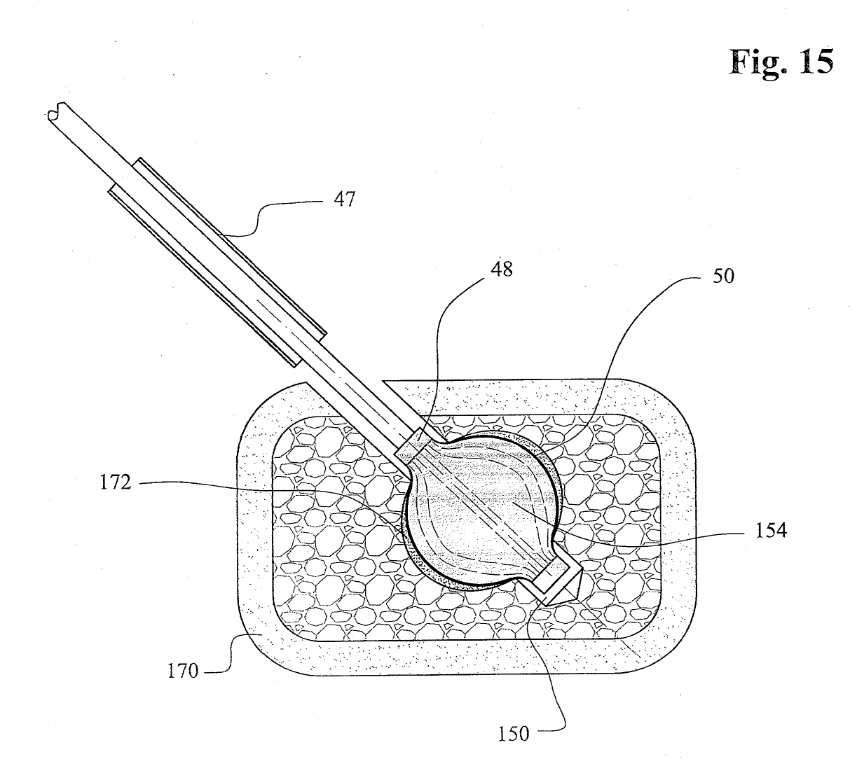

In FIG. 16, when the double-umbrella basket 48 is deployed, the particles 172 contained within the inner membrane covering 50 may be released when the outer membrane covering 53 is stretched and tears open. The perforated tear slots 52 on the outer membrane 53 are capable of bursting or tearing (the terms bursting and tearing used interchangeably herein) upon reaching a certain amount of deformation, which occurs when the double-umbrella basket 48 is deployed. The double-umbrella basket 48 then may be rotated about 20 to 30 degrees in a first direction, and then about 20 to 30 degrees in a second direction and the previously deposited particles 172 may be spread against the wall surface of the bone. In FIGS. 10, 11 and 13, for ease of illustration, particles 172 are not shown. The particles 172 may either be dry or wet, and accompanied by a liquid or carrier substance as known in the art. FIG. 10 shows the assembly of the introducer 47 and the double-umbrella basket 48 outside the surgical site. FIG. 11 shows the same assembly inserted into the surgical site 150. After the apparatus reaches within the surgical site 150, the cannula introducer 47 may be partially retracted exposing the undeployed double-umbrella basket 48 as shown in FIG. 13.

In some embodiments, the double-umbrella basket 48 may be made of surgical stainless steel or any shape memory metal alloy. Non-limiting examples of such materials are: 316L stainless steel, cobalt-chromium-molybdenum alloy, or any shape-memory alloy such as Nitinol®. The outer membrane covering 53 may be made of a relatively thin biocompatible and bioresorbable polymer. Examples of such materials include polycaprolactone and DLPLG, as described elsewhere herein, and any of various materials having known use as membranes for cardiac catheterization and similar applications.

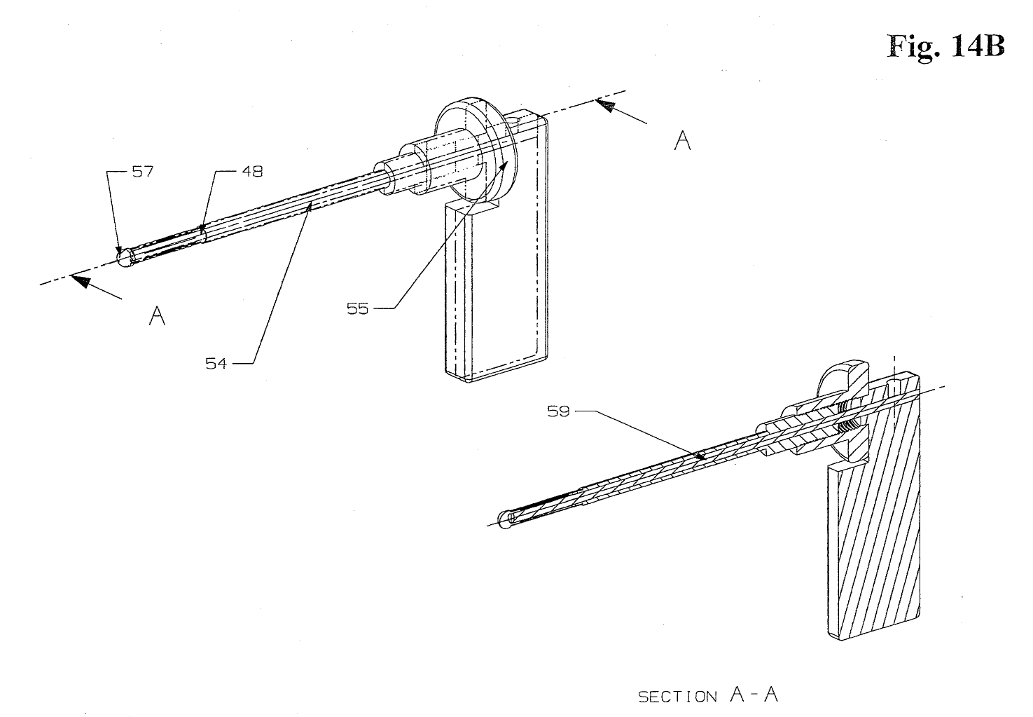

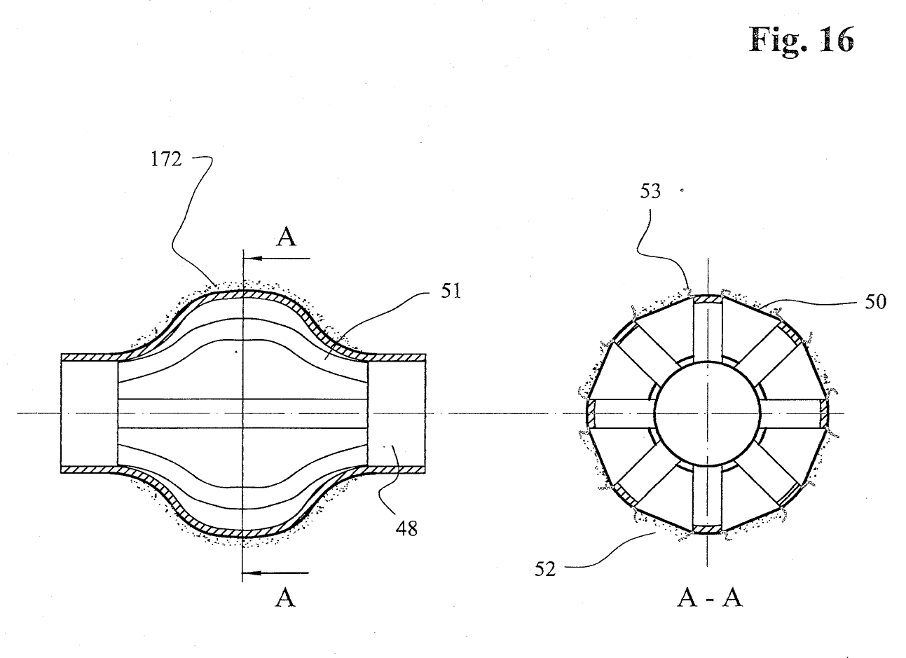

In FIG. 14A, the expandable basket 48 may be deployed by axial compression using any variety of mechanisms, such as a mechanical threaded plunger tube 54 when the knob 55 of the rod 54 is rotated. Rotating the knob 55 may move the rod 54 distally, which applies an axial compressive force onto the basket assembly. The compressive force may cause the basket assembly struts 51 to curve outward; thus, creating an end-to-end double-umbrella basket shape. Should this procedure be adequate using stainless steel, then no further deployment action may be necessary. In other embodiments, the expandable basket 48 may be made of, for example, either a shape-memory metal alloy or a cobalt-chromium-molybdenum alloy. In this embodiment, the struts 51 may be pre-formed to the desired dimensions. The desired dimensions may be heat-set to the double-umbrella basket deployed size and shape. Upon mechanical or temperature-dependent activation, the double-umbrella basket 48 deploys to the desired size and shape. The double-umbrella basket 48 may curve outward to occupy a larger volume. FIG. 15 shows the deployed expandable basket 48 to create the desired shape at surgical site 150. Upon mechanical or temperature-dependent activation, the double-umbrella basket 48 changes configuration to the desired size and shape. The double-umbrella basket 48 may retract inwardly to occupy a smaller volume sufficient for retraction into the cannula introducer 47. FIG. 17 shows the undeployed double-umbrella basket 48 removed from the surgical site 150.

Referring again to FIG. 16, once the outer membrane 53 is mechanically lysed upon deployment of the double-umbrella basket 48, the particles 172, which are contained in the inner membrane covering 50, may be pressed outward by the expansion of the basket assembly and may be deposited onto the surface of the bone wall cavity 154. After the interior surface of bone wall 154 in bone 140 has received a layer of particles 172 from the inner membrane covering 50 after the outer membrane covering 53 tears or dissolves. The basket assembly apparatus may then be rotated (as a rigid body) approximately 20 to 30 degrees in a first direction and then back 20 to 30 degrees in a second direction, preferably opposite the first direction. These rotations may somewhat evenly smooth out the particles 172.

To remove the double-umbrella basket 48 from the surgery site 150, the knob 55 of the rod 54 assembly is rotated in an appropriate direction to return the stainless steel double-umbrella basket 48 to its original undeployed size and shape. As an alternative in some embodiments, a shape-memory metal alloy double-umbrella basket may be used such that a rotation of the knob 55 of the rod 54 may axially pull the basket assembly to collapse back to its original shape given the superelasticity of the shape-memory alloy. This reversion to the original size and shape may allow the whole assembly to be withdrawn. What remains of the outer membrane covering 53 may be partially removed when the basket assembly is withdrawn from the surgical site or the outer membrane covering 53 or parts of the outer membrane 53 may be left behind inside the surgical site 150 and may be resorbed by the body over time.

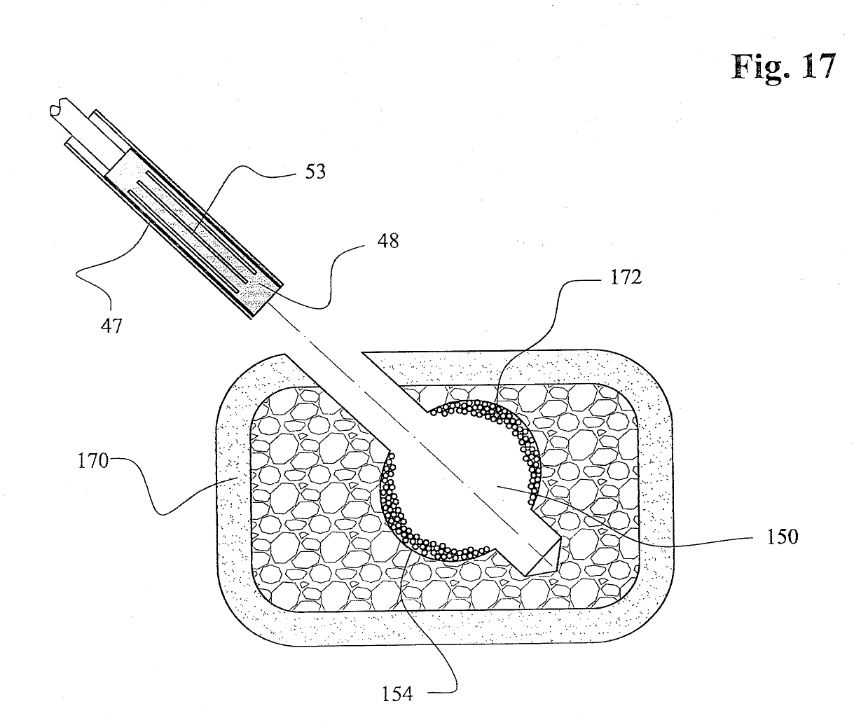

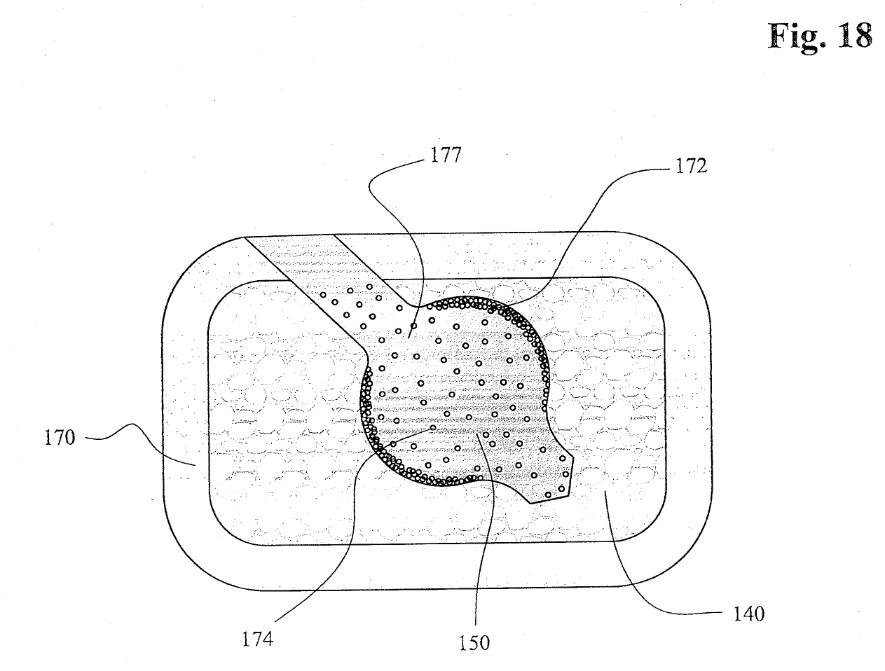

The cement precursor 177 (containing particles 174) may be prepared in the usual manner and inserted or injected into the bone cavity surgical site 154 as will be appreciated by those skilled in the art. FIG. 17 shows cannula introducer 47 and any remaining outer membrane covering 53 removed from the surgical site 150. FIG. 18 shows the surgical site 150 in bone 140 filled with a mixture of cement precursor 177 with particles 174 and particles 172. Finally, FIG. 19 shows the cement precursor 177 in bone 140 having hardened to form cement 176, containing particles 174 and particles 172.

It can be appreciated that in the above described method, if the particles 172 are deposited as a layer of particles 172, which are substantially in contact with each other having a packing fraction and if that is followed by injecting cement precursor 177 containing its own particles 174, there may be some flowing of cement precursor 177 into the interstices within the pre-placed particles 172. However, the particles 174, which were contained in the cement precursor 177, may not be able to move into the interstices between particles 172. This holding-back of some particles could result in a less-than-fully-sharp variation of particle concentration in the immediate vicinity of the interface between regions 162 and 164. Near the interface between regions 162 and 164 there may be places in which the concentration of particles is higher than it was in the cement mixture as injected, yet the concentration may not be as high as in the pre-placed particles 172. This may appear as a local gradient in the particle concentration. The particle coating 172 could retard the leakage of the composite cement outside of the bone. The leakage of the composite cement and particles may become emboli, an undesirable side effect in vertebroplasty and kyphoplasty procedures.

Lastly, the surgical incision may be closed following a surgical procedure known to one skilled in the art. FIG. 19 shows the repaired vertebra, which may be another bone in other embodiments, in which the composite has non-uniform local concentration of particles, that is, a higher particle density (=β) in the region 162 next to bone to allow more bone ingrowths to take place, and a lower particle density (=α) in the bone cement bulk 164 to provide a maximum fatigue-resistant composite to support the repaired bone. Again, it should be noted that the clear distinction between the bone-contacting region 162 and the bone cement bulk region 164 in FIG. 19 is partly for illustrative purposes. In reality, there may be a local transition in which the particle concentration in the bone cement may change gradually over a certain distance from the concentration in the bone-contacting region 162 to the concentration in the inner bulk region 164. In FIG. 19, in order to indicate a situation representing the completion of the medical treatment, what had been labeled in previous Figures as cement precursor 177 is labeled as cement 176 (having substantially completed its hardening process).

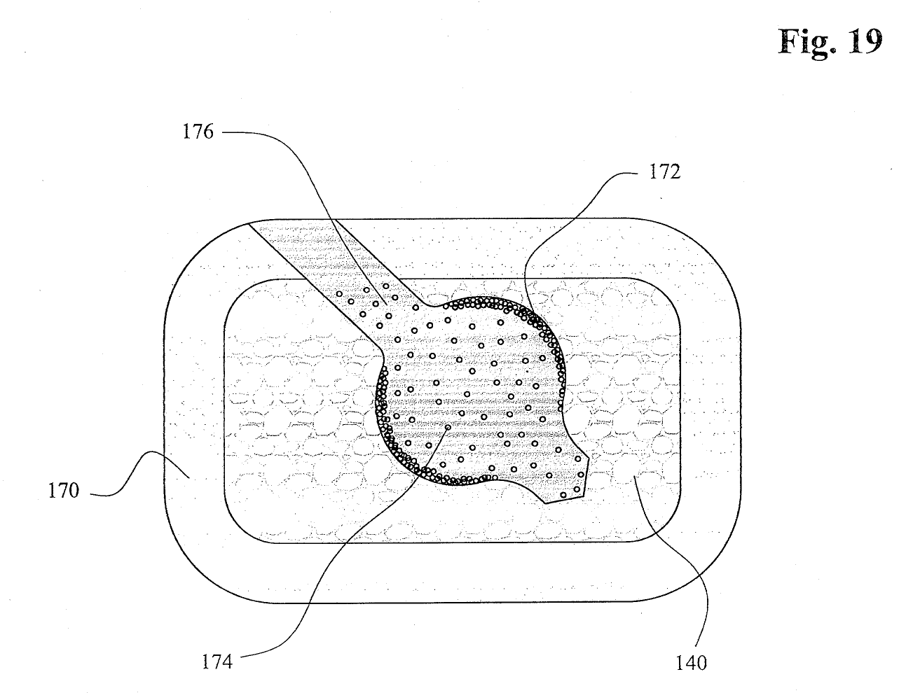

FIGS. 20 through 27 illustrate one implementation of the present invention in which the cavity-forming tool and particulate-dispensing tool are separate devices. Referring to FIG. 20, there is illustrated a lateral partial cut away view of a vertebral body, such as a lumbar vertebral body 200. As has been discussed elsewhere, the vertebral body is used as an illustrative bone and the present invention may be practiced on any of a wide variety of bones throughout the body.

The exterior of the vertebral body 200 generally comprises a superior end plate 202 and an inferior end plate 204 covering a thin wall of cortical bone. Contained within the vertebral body 200 is a cancellous bone matrix network 206.

As applied in the context of the spine, methods of the present invention may be accomplished utilizing open surgical access, or a less invasive access such as a percutaneous puncture. As illustrated in FIG. 20, an elongated access cannula introducer 208 has been percutaneously introduced into the patient and advanced through soft tissue such that a distal end 214 is positioned within the cancellous bone 206. The access cannula introducer 208 comprises an elongated tubular body 210, having a proximal end 212, a distal end 214 and a central lumen 216. Preferably, the distal end 214 is provided with a sharpened tip or trocar, such as a single, double or triple bevel, as is understood by those skilled in the art. Access cannula introducer 208 preferably comprises a medical grade material such as surgical steel; although any of a variety of materials having suitable physical characteristics may be utilized. The proximal end 212 may be provided with a proximal hub 218, to facilitate handling and also to optionally allow releasable engagement with various tools adapted to extend through central lumen 216.

The access cannula introducer 208 may be advanced through soft tissue on the back of the patient and into the vertebral body 200. The propagation axis for introduction of access cannula introducer 208 may be transpedicular, although other approaches such as lateral, posterior lateral, extrapedicular and/or anterior may be used depending upon the level of the spine treated and/or the intervening anatomical features as is understood by those of skill in the art. Depending upon the gauge of the access cannula introducer 208, an internal obturator or stylet (not illustrated) may be removably positioned within the central lumen 216, as is understood in the art. Preferably, once tubular access cannula introducer 208 has been positioned as illustrated in FIG. 20, it will provide access to the interior of the vertebral body for the remainder of steps in the procedure.

During insertion of the access cannula introducer 208, the location of the cannula introducer 208 may be monitored using any of a variety of visualization equipment such as fluoroscopy (i.e., real time X-ray), ultrasound, CT scanning equipment, MRI, or other monitoring equipment commonly used including computer aided guidance and mapping equipment.

In one implementation of the invention, the distal end 214 of the access cannula introducer 208 is positioned in the vertebral body 200 at a location towards the posterior side of the vertebral body 200. The distal end 214 may alternatively be positioned in any of a variety of locations throughout the vertebral body 200, such as towards the anterior side.

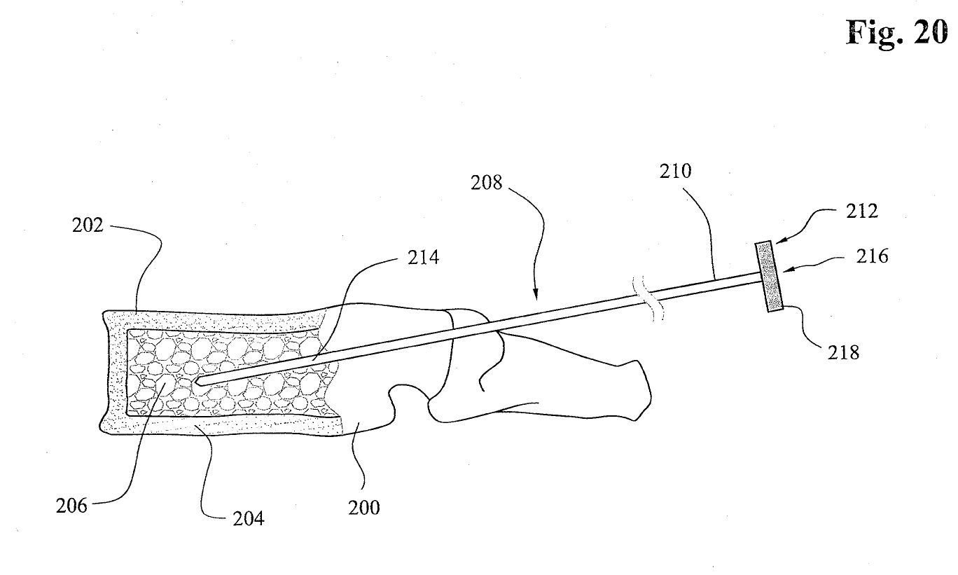

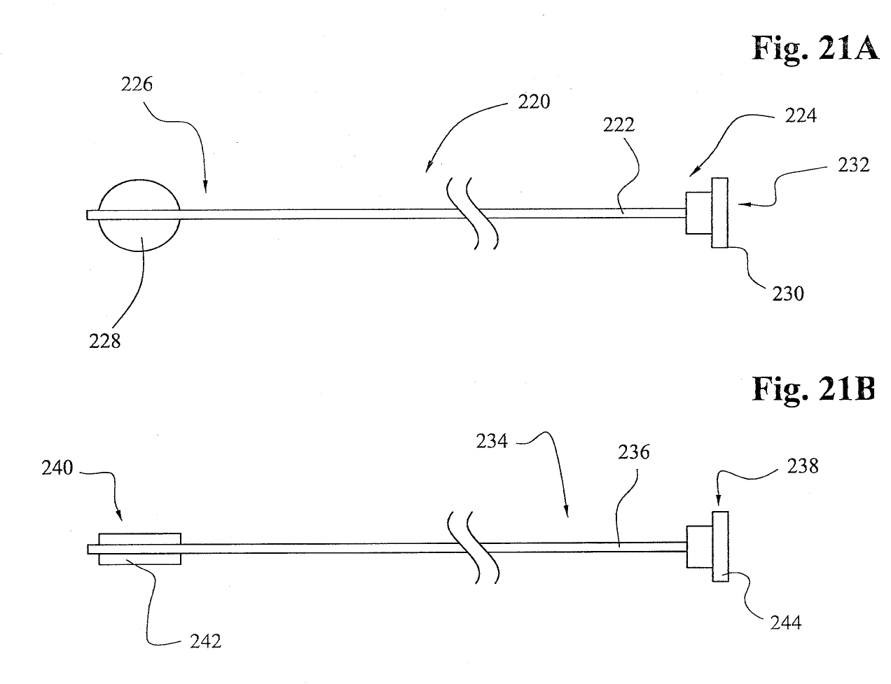

Referring to FIG. 21A, there is schematically illustrated a cavity forming tool 220 in accordance with the present invention. Cavity forming tool 220 comprises an elongated body 222 having a proximal end 224, a distal end 226 and a cavity forming element 228 on the distal end 226. A proximal hub 230 may be provided as will be appreciated by those of skill in the art.

The tools described herein may be made from any of a variety of materials well known in the medical device arts, and have dimensions that will be optimized for the specific intended target bone. In the present example, the access cannula introducer 208 may have a length within the range of from about 7 cm to about 35 cm, and an outside diameter of no greater than about 12 mm, and, in certain embodiments, no greater than about 7 mm. The associated instrumentation will be dimensioned to cooperate with the length and diameter of the central lumen 216, as will be appreciated by those of skill in the art.

The cavity forming tool 220 is dimensioned to extend axially through the central lumen 216 on the access cannula introducer 208, to access cancellous bone 206. The cavity forming element 228 may be any of a variety of cavity forming elements such as those described elsewhere herein. For example, cavity forming element 228 may comprise a single walled inflatable balloon, a double walled inflatable balloon, a double-umbrella mechanical deployable basket, or other mechanical expansion elements, each of which can be utilized to form a cavity by bone compaction. Alternatively, the cavity can be formed by removal of bone. This may be accomplished using any of a variety of cutters, burrs or brushes which may be manipulated within the vertebral body 200 to form the cavity. Cavity formation by removal of material may also be accomplished by or assisted by the introduction of any of a variety of chemical or biochemical agents, such as enzymes, acids or other materials that reduce or eliminate cancellous bone.

As a further alternative, the cavity forming element 228 may comprise any of a variety of transducers or sources of energy, such as radio frequency (RF) electrodes, microwave or high intensity focused ultrasound (HIFU) transducers, heat sources or cryogenic cooling chambers, which may be utilized to disrupt and facilitate removal of selected portions of cancellous bone. In the illustrated embodiment, the cavity forming element 228 is an inflatable balloon, in communication with the proximal end 224 of the body 222 by an inflation lumen 232. The inflatable balloon may be folded and provided with a lubricous coating or other feature to facilitate axial advance through the central lumen 216 in the access cannula introducer 208 while the inflatable balloon is in a deflated profile.

Expansion of the cavity forming element is preferably accomplished to a sufficient degree that a cavity of the desired size will remain following removal of the cavity forming tool 220. In the embodiment illustrated, the inflation volume of the inflatable balloon is generally at least about 0.2 cc, but may be greater such as at least about 1, 2, 4, 6 or 8 cc or more depending upon the bone quality and density. In addition, the size of the inflated balloon as well as the shape of the balloon will be influenced by the nature of the bone in which the treatment is to be accomplished. For treatment in a vertebral body, a spherical balloon or a cylindrical balloon may often be used. However, for treatment in the proximal femur, for example, an elongated cylindrical, or a frusto conical shaped balloon or a non-regular geometric shaped balloon may be utilized to accommodate the irregular shape of the medullary canal.

Any of a variety of alternative cavity forming devices may also be used, such as any of those disclosed in U.S. Pat. No. 6,726,691 to Osorio et al., entitled Methods for Treating Fractured and/or Diseased Bone, the entirety of which is incorporated by reference herein.

FIG. 21B schematically illustrates a particulate-dispensing tool 234. In general, the particulate-dispensing tool 234 includes an elongated tubular body 236, having a proximal end 238 and a distal end 240. The distal end 240 is provided with a dispensing head 242, which may be any of a variety of structures for dispensing particulate as has been disclosed elsewhere herein. For example, dispensing head 242 may be an inflatable balloon having a plurality of perforations. Alternatively, the dispensing head 242 may comprise a double walled balloon with particulate entrapped between the walls, in which the outer walled balloon is rupturable in situ. Alternatively, the dispensing head 242 may comprise a plurality of apertures along the sidewall of tubular body 236, for releasing particulate material within the bone.

Particulate dispensing tool 234 is preferable additionally provided with a proximal hub 244, for coupling to a source of particulate material. The nature of the dispensing head 242 and the proximal hub 244 may be varied widely, depending upon the compositional nature of the particulate (e.g., dry powder, gel, slurry, paste, etc.) to be dispensed as has been discussed elsewhere herein.

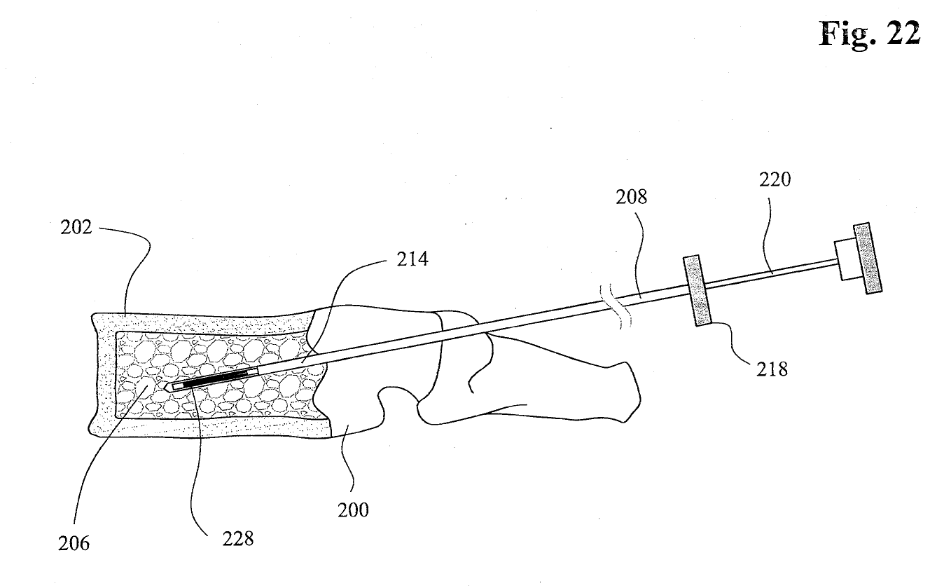

Referring to FIG. 22, the cavity forming tool 220 has been coaxially advanced through the central lumen 216 on access cannula introducer 208, to position the cavity forming element 228, while in a deflated or reduced profile configuration, within cancellous bone 206.

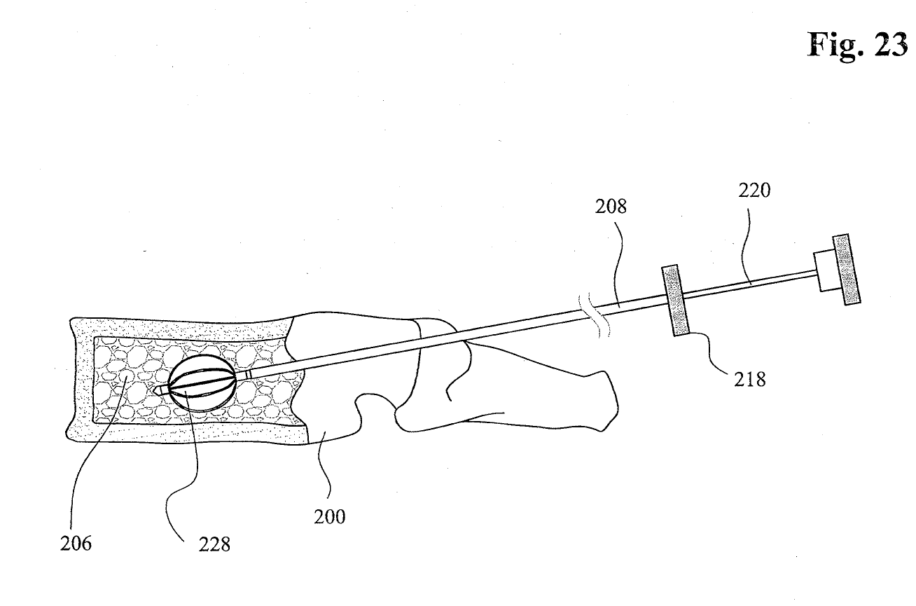

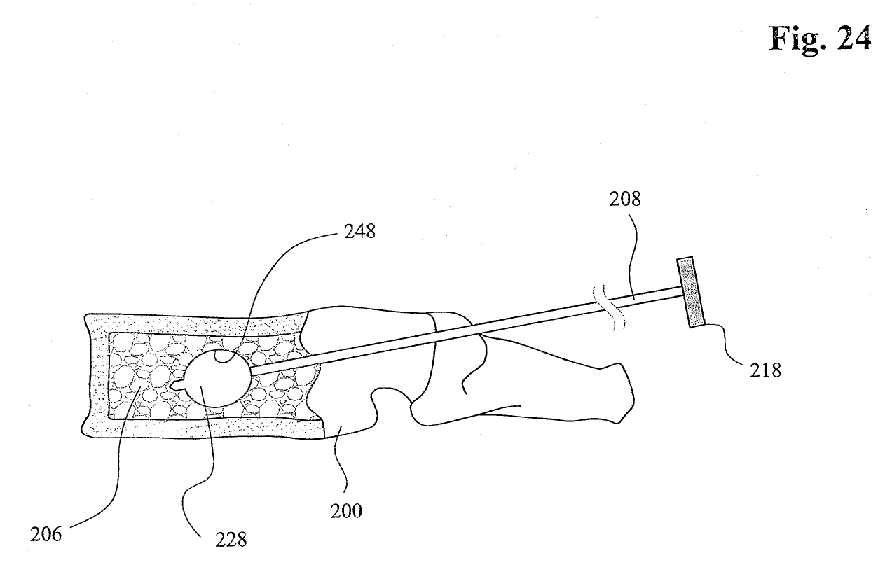

Referring to FIG. 23, the cavity forming element 228 has been transformed to its enlarged profile, to compact cancellous bone 206 and create a cavity 246. In FIG. 24, the cavity forming tool 220 has been removed while the access cannula introducer 208 remains in position to provide access to the cavity 246 for subsequent steps in the procedure.

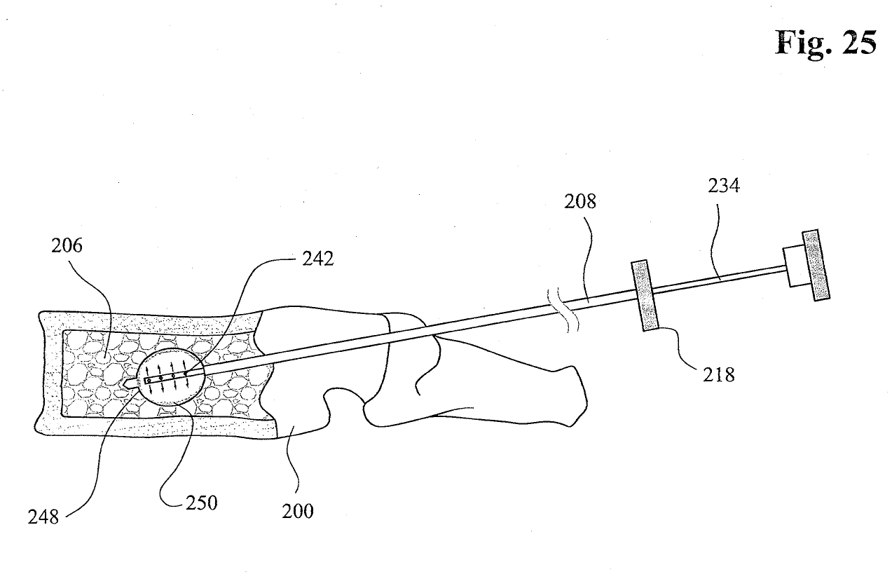

Referring to FIG. 25, the particulate-dispensing tool 234 has been coaxially advanced through the central lumen 216 of access cannula introducer 208, to position the dispensing head 242 within the cavity 246. As illustrated in FIG. 25, particulate is being dispensed from the dispensing head 242, to provide a lining or coating 250 on the wall 248 of the cavity 246 as has been discussed herein. The particulate may be in the form of a dry powder, gel, paste, or other flowable form as has also been discussed herein.

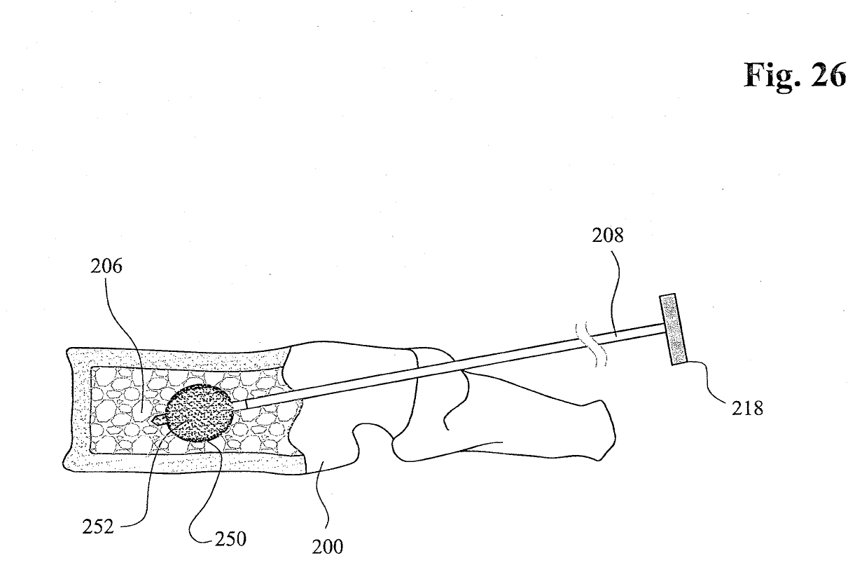

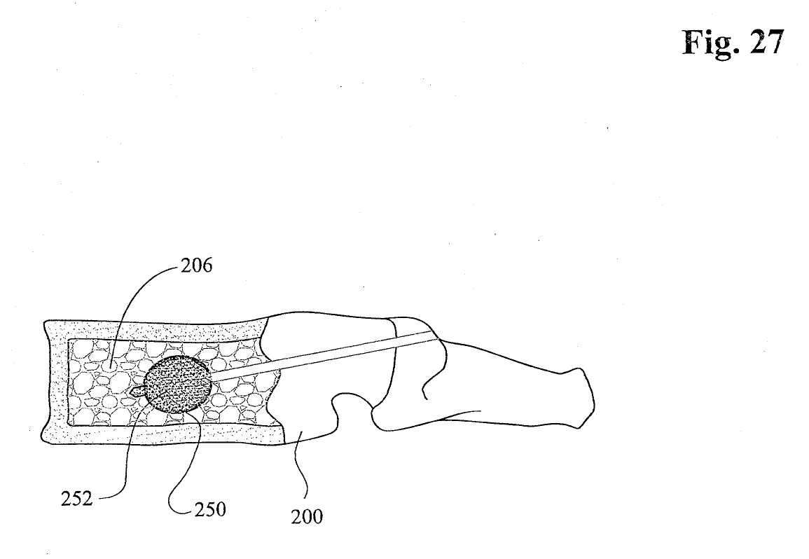

Referring to FIG. 26, the particulate dispensing tool 234 has been withdrawn from the access cannula introducer 208. Thereafter, a source of a hardenable media such as PMMA or others discussed elsewhere herein, having a particulate blended therein to form the filler material is coupled to the proximal hub 218 and the hardenable media is advanced through the central lumen 216 to at least partially fill and preferably completely fill the cavity 246 which remains following introduction of the particulate, to form the composite implant 252. Referring to FIG. 27, the access cannula introducer 208 may thereafter be removed. Removal of the access cannula introducer 208 may be accomplished immediately following introduction of the filler, or may be accomplished following a period of time in which the hardenable filler media begins to at least partially harden in order to minimize the risk of escape of non-hardenable or hardenable media through the access tract. The access tract may thereafter be closed in accordance with known techniques or simply left to heal, depending upon the diameter and desired clinical result as known to those skilled in the art.

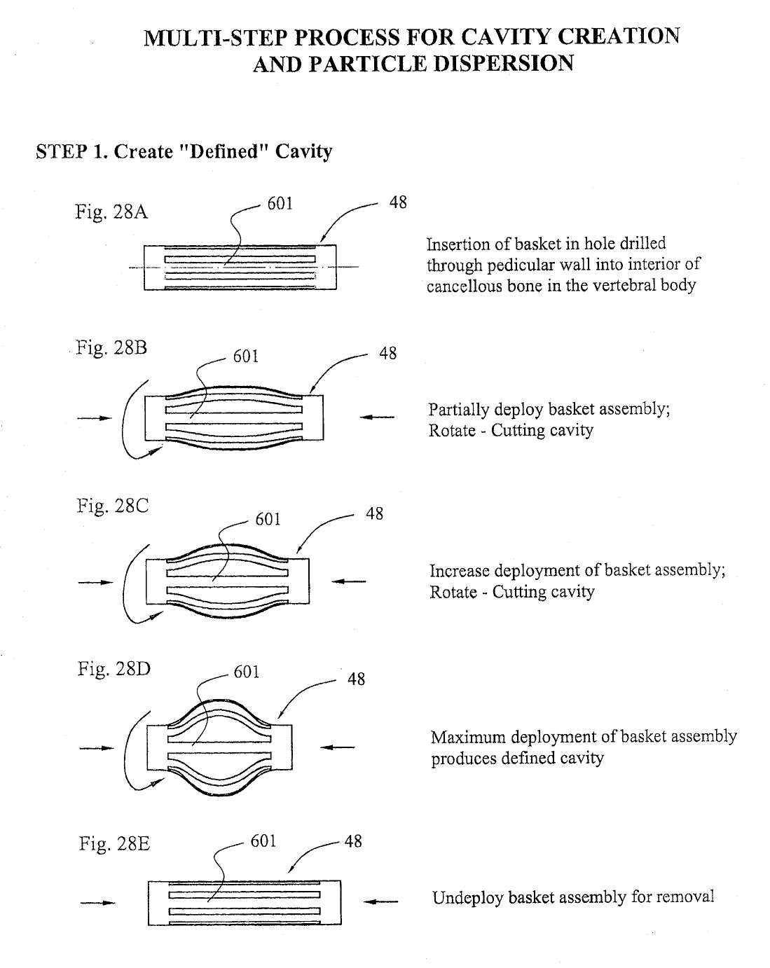

FIGS. 28 through 30 illustrate one implementation of the present invention in which the cavity-forming tool, the particulate-dispensing tool, and the particulate-spreading tool are three separate devices and the method of their use includes multiple steps.

The cavity-forming tool, which preferably has an insertable length of between about 1.5 cm to 3.0 cm may comprise a plurality of flexible struts 51, preferably having sharp edges. In this embodiment of the cavity-forming tool, as shown in FIGS. 28A through 28E, the cavity-forming tool is a variation of the double-umbrella basket 48. The double-umbrella basket 48 cavity-forming embodiment may be made of surgical stainless steel or any shape-memory metal alloy. Examples of such materials are: 316L stainless steel, cobalt-chromium-molybdenum alloy, or any shape-memory alloy such as Nitinol®

In this example of the multiple-step method, the double-umbrella basket 48 cavity-forming tool acts within the interior of the cancellous bone of a vertebral body by axial force.

The tool, in some embodiments, preferably includes a center rod 59 with integral distal endcap 57 that is coaxial with outer sleeve 54, double-umbrella basket 48, and knob 55. The distal end of the outer sleeve 54 preferably abuts the double-umbrella basket 48 assembly. The proximal end of the sleeve 54 preferably abuts the knob 55 component. The distal end of the double-umbrella basket 48 can abut the center rod 59 and distal endcap 57. The double-umbrella basket 48 cavity-forming tool may be keyed to the center rod 59 to prevent spinning, as shown in FIG. 14B, such that a rotation can be applied from the proximal end of the device when the knob 55 is rotated. Rotating the knob 55 may move the outer sleeve 54 distally against the double-umbrella basket 48. As the double-umbrella basket 48 moves distally against the center rod 59 distal endcap 57, it may create an axial force onto the expandable basket 48 assembly. Alternatively, the device can be actuated by a ratchet or other mechanism as known in the art.

The axial force may cause the basket assembly of the cavity-forming struts 51 to curve outward against the cancellous bone matrix; thus, creating an end-to-end double-umbrella basket shape when fully deployed as shown in FIG. 28D.

The double-umbrella basket 48 cavity-forming tool is partially deployed as shown in FIG. 28B and may be rotated to cut the cancellous bone matrix and create a cavity in the cancellous bone. Subsequent additional axial force results in further deployment of the double-umbrella basket 48 cavity-forming tool as shown in FIGS. 28C and 28D. The double-umbrella basket 48 cavity-forming tool may again be rotated to cut more cancellous bone and further increase the size of a cavity in the cancellous bone. This method of increasing the size of the double-umbrella basket 48 cavity-forming tool may be repeated as many times as required to produce the desired cavity shape and may be referred to as the “cavity calibration system”.

Once the desired cavity shaped is formed in the cancellous bone in this example use of the invention, the double-umbrella basket 48 cavity-forming tool may be undeployed and restored to its original tubular shape by removing axial force as shown in FIG. 28E. The double-umbrella basket 48 proximal and distal ends may be anchored or fixed to their respective abutments for undeployment if required (basket material dependant). The double-umbrella basket 48 cavity-forming tool may then be withdrawn from the cancellous bone through the cannula introducer 47 as previous described herein.

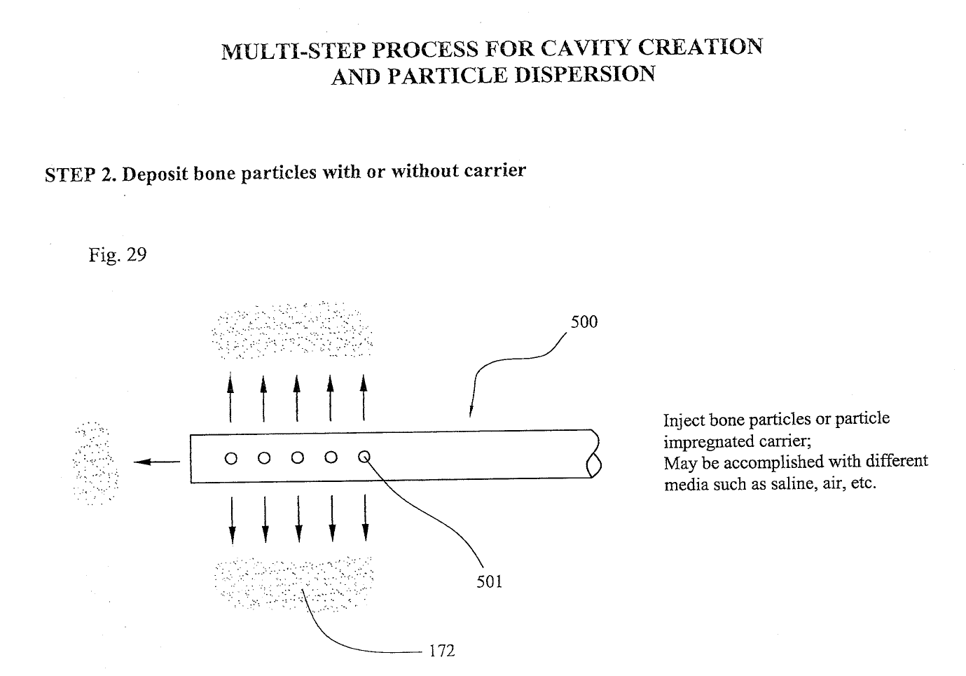

In this example of the multiple-step method, a second particulate-dispensing device 500 shown in FIG. 29 may be used to deliver particles 172. The distal end of the particulate-dispensing device may contain holes 501 to allow power-assisted or manual injection of particles 172. The delivery medium for the particles 172 may be in the carrier form of a gel, paste, slurry, saline, air, or other methods known to those skilled in the art. Other agents may also be included either to modify the viscosity of the particles 172 carrier or to modify other characteristics of the cavity and the procedure, including thrombin, fibrinogen or other thrombogenic agents; enzymatic substances such as hyaluronidase or other lytic substances; osteogenesis-enhancing agents; alternatively or simultaneously antibiotics may be included.

In this example of the multiple-step method, a third particle-carrier-spreading device 600 may be used to spread the particles 172 that are disbursed by the particulate-dispensing device 500. Such particle-carrier spreading may be accomplished with a polymer-covered double-umbrella basket 48, a balloon, mechanical wipers, or other devices available to those skilled in the art of this type of application. The particle-spreading device 600 may be deployed and rotated as much as required, such as, for example, at least about 10, 20, 30, 40, 50, 60, or more degrees to dispense the particles 172 or the particle-impregnated carrier.

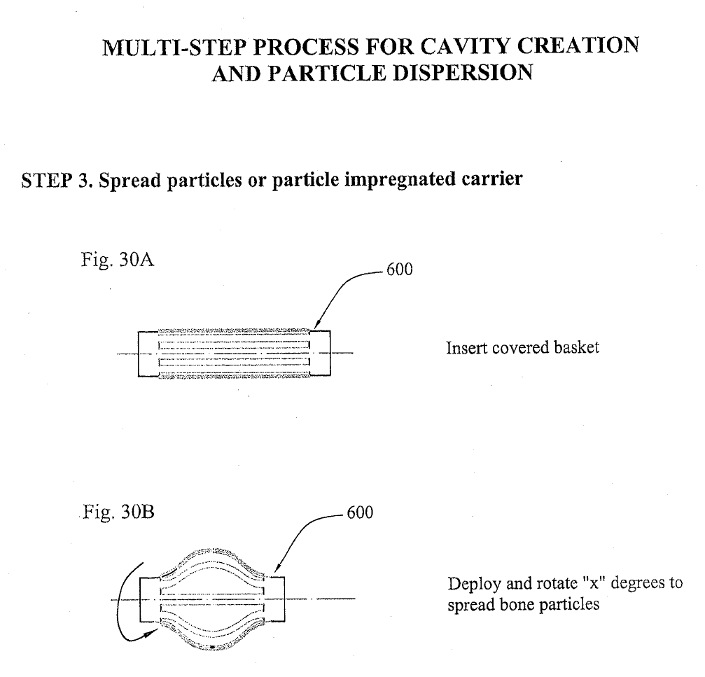

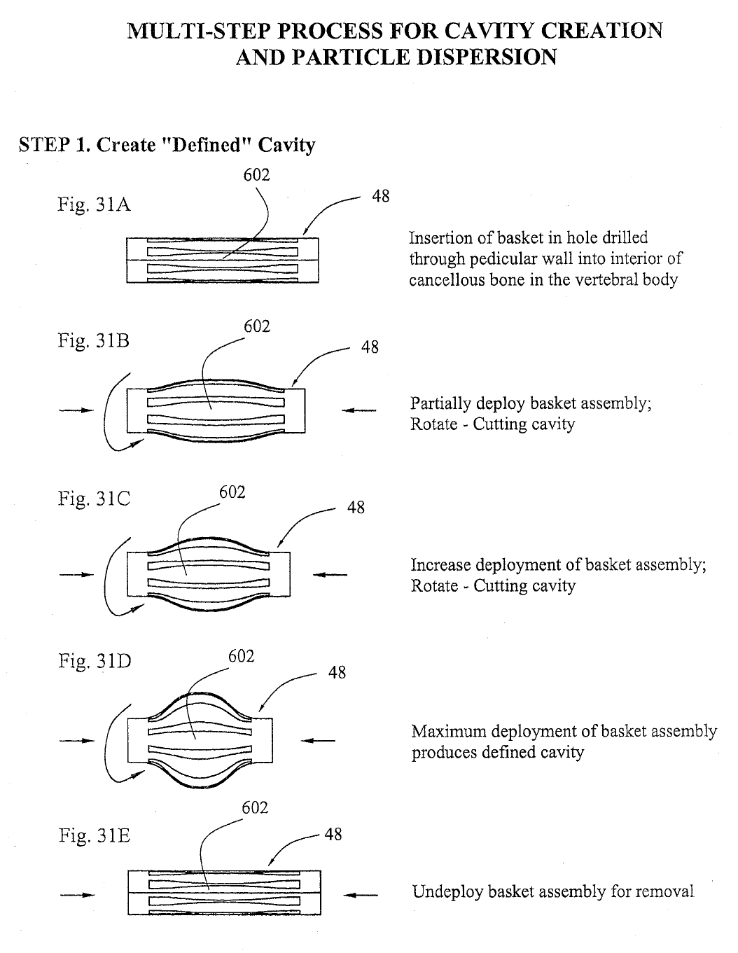

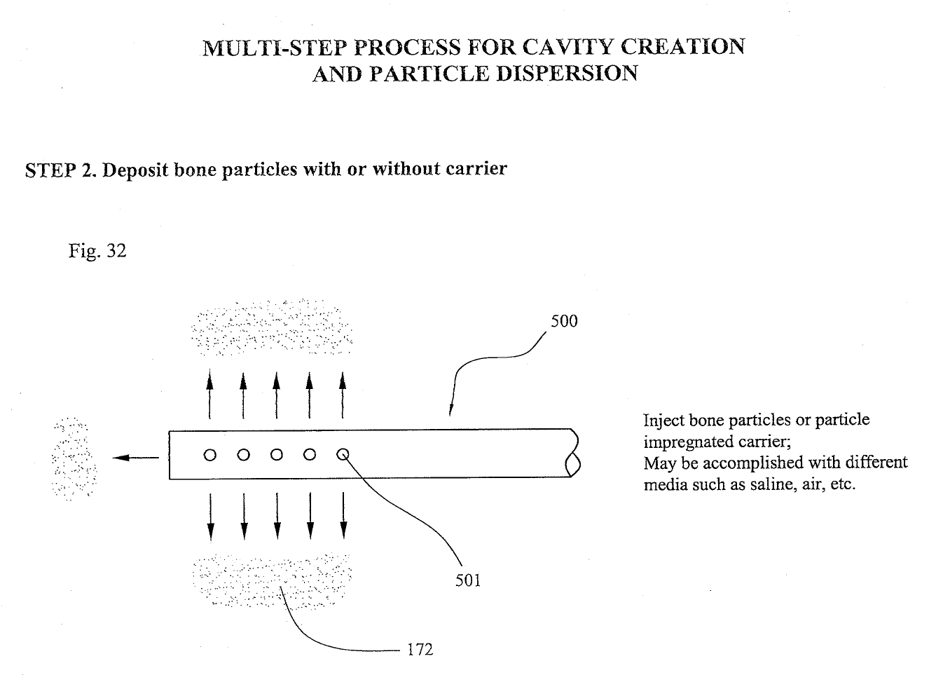

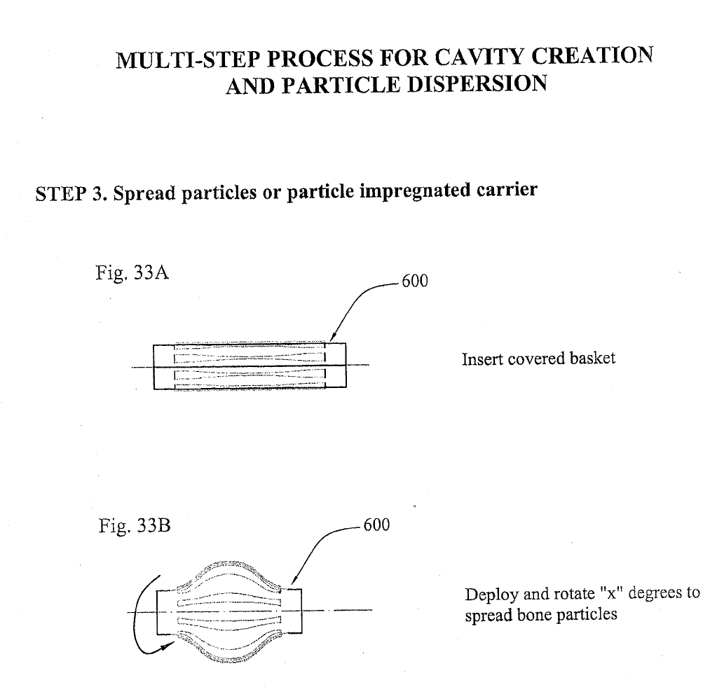

FIGS. 31-33 generally illustrate steps of creating a bone cavity and dispersing particles within the cavity as shown and described in connection with FIGS. 28-30. However, the basket 48 shown in FIGS. 31A-E and FIGS. 33A-B have generally “reversed bow-tie” shaped struts 602 instead of generally rectangular struts 601 as illustrated in FIGS. 28A-E and FIGS. 30A-B. As illustrated in FIGS. 31A-E and 33A-E, the “reversed bow-tie” struts 602 decrease in width from a first end of the strut 602 to a midportion, and then increase in width from the midportion to a second end of the strut 602. This strut 602 configuration can advantageously provide increased strength and stability at the strut midportion. One of ordinary skill in the art will recognize that a wide variety of other strut configurations, for example, struts with barbs anywhere along the length of the struts, struts with undulating widths, and the like.

Method of Use

The bone cement composite with non-uniform concentration of particles, of the present invention, can be used in the implantation of prostheses, in the repair of vertebral fractures (compression fractures due to osteoporosis or trauma) or in vertebroplasty or corrective surgeries for hump-back (kyphosis) called kyphoplasty. In oncology or cancer cases, the bone cement of the present invention can be used to treat various diseases and disorders, for example, multiple myeloma and primary or metastatic tumors of bone including sarcomas, lung, colon, prostate, breast and thyroid cancer, among others (e.g., bone metastatic lesions arising from cancer of the lung, breast, and lymph nodes.) Benign lesions including, for example, giant cell tumors and hemangioma are also treatable using the gradient system provided by the bone cement composite with differential impregnated particle density of the present invention. It should be appreciated, however, that the bone cement so described, in accordance with the invention, is not limited in its applications to small bones, e.g., vertebrae. The composite bone cement of the present invention is also applicable to the treatment of diverse bone disorders either in the major musculoskeletal joints or the diaphysis of long bones. Furthermore, one of ordinary skill in the art will recognize that the composite and methods herein also can be used, or adapted to, for example, enhance a bone to implant bond; enhance a bone to bone bond; roughen the surface of a formed in situ implant; enhance bone ingrowth of an implant; or facilitate cancellous bone integration. Methods of creating cavities and filling the cavity with the disclosed composite can also take place in vitro or ex vivo.

From the standard ASTM fatigue test, for a selected case, we have determined that approximately 30% weight fraction of inorganic bone particles well-mixed during the doughing period of commercial PMMA yielded the optimum crack initiation resistance and fatigue life for the standardized fatigue specimen, see Y. K. Liu, J. B. Park, G. O. Njus, and D. Stienstra, “Bone-particle-impregnated bone cement: An in vitro study,” Journal of Biomedical Materials Research, Vol. 21, 247-261, 1987; and in H. C. Park, Y. K. Liu and R. S. Lakes, “The material properties of bone-particle impregnated PMMA,” Journal of Biomechanical Engineering, Vol. 108, 141-148, 1986.

Introducing such a composite into a total joint replacement application, the in vivo weight-bearing canine experiments conducted by K. R. Dai, Y. K. Liu, J. B. Park, C. R. Clark, K. Nishiyama, Z. K. Zheng, “Bone-particle-impregnated bone cement: An in vivo weight-bearing study,” Journal of Biomedical Materials Research, Vol. 25, 141-156, 1991 showed that there were sufficient bone ingrowths into the cement to increase the interfacial shear strength by a factor of 3.6. However, as the composite cement volume decreases, the numbers of particles in contact with living bone decreases. To increase the interfacial shear strength, one must increase the volume fraction of particles near the surface. For example, some experiments have shown that bone ingrowth need not penetrate more than about 2 mm to obtain its maximum shear strength. Hence, for a small volume application, e.g., vertebroplasty or kyphoplasty, one can calculate or estimate the volume of the particles to occupy a suitable volume fraction of 2 mm thick surface spheroid.

The appropriate volume of particles needed to occupy the 2 mm thick surface spheroid is a function the particles used and the volume of material and the interaction between the particles and the material.

The composite and methods of the present invention can advantageously improve a bone to implant bond, for example, the bond of a bone to a joint replacement, rod, screw, other fixation devices, and the like. A bone to implant cemented prosthesis has two vulnerable interfaces: 1) cement to prosthesis, and 2) cement to bone. The first vulnerability can be solved, for example, by pre-coating the stem of the prosthesis with an acrylic cement under industrial laboratory conditions which will make that interface as strong as is possible (see J. B. Park and R. S. Lakes “Biomaterials: An Introduction—Second Edition,” Plenum Press, 1992, pp 324-328), When the acrylic cement dough is introduced in the intermedullary canal under pressure and with any air bubbles removed by a wire mesh, the pre-coated stem is then seated through the polymerization of the old cement with the new cement. The second vulnerable interface is solved by the present invention, that is, the living bone grows into the composite cement bulk because the presence of the particulate composite on the vertebral wall surface elicits either osteoconduction or osteoinduction or both, as described above.

Although the cemented implant is the gold standard in total joint replacement, the implant lasts, on average, ten years before loosening at either of the aforementioned vulnerable interfaces. The success rate for cemented prostheses is outstanding for the hip and very good for the knee prior to loosening. Above all, because the cement acts as a grout, it is surgically forgiving. Should a revision surgery be necessary, then removal of the prosthesis would require ablation of substantial bone mass; thus, making the revision surgery more difficult. If the patient were not approaching older age, then a non-cemented prosthesis should be used. However, in this cement-less embodiment, the stem is sintered with beads of the same material as the stem to create interconnected optimum size pores for bone ingrowth. However, the success of the procedure demands that the surgeon be precise in placement of the prosthesis via interference fit. If the fit is too tight, it will cause bone necrosis or shattering of osteoporotic bone; if not tight enough, it will loosen soon after surgery. Furthermore, weight bearing is not allowed until much later postoperatively when compared to the cemented implant. If a revision surgery is needed for the cement-less prosthesis, than removal of the prosthesis would take away only a smaller portion of the cancellous bone thus allowing for an easier revision surgery as known to those skilled in the art.

Further Comments

An embodiment of the present invention includes differential composite bone cement whose interior bulk volume potentially has excellent fatigue properties under in vivo cyclic loading conditions. The presence of particles in the bulk of the composite potentially has a fatigue life that is approximately one order of magnitude longer than the fatigue life of pure PMMA bone cement. The fatigue life is determined by the number of load or displacement cycles experienced by the specimen before it fails. In some embodiments, the fatigue life can be at least about 20 years, 30 years, 40 years, 50 years, or more.

At the same time, the composite of the present invention may provide for very good bone ingrowth at the bone-cement interface, thereby providing much improved interfacial shear strength and rigidity.

While this invention has been particularly shown and described with references to embodiments thereof, it will be understood by those skilled in the art that various changes in form and details may be made therein without departing from the scope of the invention. For all of the embodiments described above, the steps of the methods need not be performed sequentially.

What is claimed is:

1. A composite comprising:
(a) bone cement; and
(b) particles contained in said bone cement,
wherein said composite has at least two different non-zero local particle concentrations of said particles, said local particle concentrations being controlled to have desired values at desired locations.

2. The composite of claim 1, wherein said composite immediately adjacent to a bone has a particle concentration greater than a particle concentration away from said bone.

3. The composite of claim 1, wherein said bone cement is substantially non-bioresorbable.

4. The composite of claim 1, wherein said particles are substantially bioresorbable.

5. The composite of claim 1, wherein said composite is contained within a cavity in a bone of a patient.

6. The composite of claim 1, wherein said composite exhibits a gradient of the local concentration of said particles.

7. The composite of claim 1, wherein said particles comprise at least one substance selected from the group consisting of: inorganic bone; demineralized bone; natural bone; bone morphogenic protein; collagen; gelatin; polysaccharides; polycaprolactone (PCL); polyglycolide (PGA); polylactide (PLA); DLPLG which is a copolymer of PLA and PGA; polyparadioxanone (PPDO); other aliphatic polyesters; polyphosphoester; polyphosphazenes; polyanhydrides; polyhydroxybutyrate; polyaryetherketone; polyurethanes; magnesium ammonium phosphate; strontium-containing hydroxyapatite; beta tricalcium phosphate; other forms of calcium phosphate; and carbon.

8. The composite of claim 1, wherein said bone cement comprises a substance selected from the group consisting of: polymethylmethacrylate (PMMA); hydroxyethyl methacrylate (HEMA); polyalkanoate; polyetherurethane; polycarbonate urethane; polysiloxaneurethane; and polyfluoroethylene.

9. A composite comprising:
(a) a first region comprising bone cement containing a first non-zero concentration of first particles; and
(b) a second region comprising bone cement containing a second non-zero concentration of second particles, said second concentration of said second particles being different from said first concentration of said first particles said regions being controlled to occupy desired locations.

10. The composite of claim 9, wherein said first region touches a bone and said second region substantially does not touch said bone, and wherein said first concentration of said first particles is greater than said second concentration of said second particles.

11. The composite of claim 9, wherein said first particles and said second particles are substantially identical to each other.

12. The composite of claim 9, wherein said first particles and said second particles are different from each other in some respect.

13. The composite of claim 9, further comprising additional first particles that are only partially contacted by said bone cement.

14. The composite of claim 9, farther comprising additional first particles that are not contacted by said bone cement.

15. The composite of claim 9, further comprising a gradient-containing region between said first and second regions.

16. A composite comprising:
(a) bone cement; and
(b) particles contained in said bone cement,
wherein said composite has, at least some places that are within approximately 2 mm of a bone, has a local weight fraction of particles greater than approximately 60%, and said composite more than approximately 2 mm away from said bone has a local weight fraction of particles less than approximately 40%.

17. A composite comprising:
(a) bone cement; and
(b) particles contained in said bone cement,
wherein said composite, at least some places that are within approximately 2 mm of a bone, is in a bone-ingrowth regime, and said composite more than approximately 2 mm away from said bone is in a crack-arresting regime.

18. A composite comprising:
(a) bone cement; and
(b) particles contained in said bone cement,
wherein in any substantially equiaxial local space containing more than three of said particles there can be defined a local particle concentration, and wherein said composite exhibits a gradient of said local particle concentration.

19. A stabilized bone comprising:
(a) a bone having a cavity;
(b) a composite in said cavity, said composite comprising bone cement and particles contained within said bone cement, said particles having different particle concentrations at different places within said composite;
wherein said particle concentration immediately adjacent to bone material of said vertebra or other bone is greater than said particle concentration away from said bone material of said bone.

20. The bone of claim 19, wherein the bone is a vertebra.

21. A stabilized bone comprising:
(a) a bone having a cavity;
(b) a composite in said cavity, said composite comprising bone cement and particles contained within said bone cement, said particles having different particle concentrations at different places within said composite;
wherein said particle concentrations are controlled to have desired values at defined places.

22. The bone of claim 21, wherein the bone is a vertebra.

23. A method for filling a bone cavity with a particle impregnated bone cement composite comprising:
(a) depositing bioresorbable first particles on an interior surface of said cavity; and
(b) depositing into remaining space in said cavity a cement precursor which comprises second particles.

24. The method of claim 23, wherein a first concentration of said first particles on said surface is greater than a second concentration of said second particles in said cement precursor.

25. The method of claim 23, wherein depositing said first particles comprise at least one substance selected from the group consisting of: inorganic bone; demineralized bone; natural bone; bone morphogenic protein; collagen; gelatin; polysaccharides; polycaprolactone (PCL); polyglycolide (PGA); polylactide (PLA); DLPLG which is a copolymer of PLA and PGA; polyparadioxanone (PPDO); other aliphatic polyesters; polyphosphoester; polyphosphazenes; polyanhydrides; polyhydroxybutyrate; polyaryetherketone; polyurethanes; magnesium ammonium phosphate; strontium-containing hydroxyapatite; beta tricalcium phosphate; other forms of calcium phosphate; and carbon.

26. The method of claim 23, wherein said cement precursor comprises at least one substance selected from the group consisting of: polymethylmethacrylate (PMMA); hydroxyethyl methacrylate (HEMA); polyalkanoate; polyetherurethane; polycarbonate urethane; polysiloxaneurethane; and polyfluoroethylene.

27. The method of claim 23, wherein depositing said first particles comprises depositing said first particles using an introducer and an expandable basket.

28. The method of claim 27, wherein depositing said first particles is accomplished using an expandable basket which comprises a perforated membranous covering on the basket.

29. The method of claim 27, wherein depositing said first particles using said expandable basket comprises bursting a perforated outer covering on the basket.

30. The method of claim 27, wherein depositing said first particles using said basket comprises bursting a perforated membranous covering while said covering is inside said bone cavity.

31. The method of claim 23, further comprising, at any stage during the method, causing deformation of said cavity due to pressure of deploying or partially deploying an expandable basket inside said cavity.

32. An apparatus for delivering and depositing particles on a wall of a bone cavity, the apparatus comprising a double-umbrella basket, said double-umbrella basket configured to expand by mechanical force or pressure and when expanded releases said particles.

33. The apparatus of claim 32, wherein said particles are biocompatible and bioresorbable particles.

34. The apparatus of claim 32, wherein said perforated outer membranous covering on said double-umbrella basket is capable of bursting due to the pressure or force exerted on said double-umbrella basket.

35. A method of treating a bone, comprising:
introducing within said bone a cement comprising particles, wherein said particles comprise an osteoinductive substance.

36. The method of claim 35, wherein said osteoinductive substance is also osteoconductive.

37. A method of treating a bone, comprising introducing within the bone a cement comprising particles, wherein said particles comprise an active pharmaceutical ingredient.

38. The method of claim 37, wherein the active pharmaceutical ingredient comprises an anti-neoplastic drug.

39. A method of creating a cavity, comprising:
creating an access;
introducing into said access an apparatus in a first configuration;
expanding the apparatus within the access to create the cavity;
contracting or fully contracting the apparatus within the cavity;
rotating the apparatus within the cavity;
reexpanding the apparatus within the cavity; and
fully contracting the apparatus to facilitate removal of the apparatus from the cavity.

40. The method of claim 39, wherein rotating the apparatus comprises rotating by an angle which is less than about at least about an integer multiple of a spacing angle between adjacent struts of the apparatus.

41. The method of claim 39, further comprising, after reexpanding the apparatus within the cavity, further expanding the apparatus to a greater extent.

42. A method of creating a cavity within a bone, comprising:
creating an access hole;
introducing into the access hole an apparatus in a contracted configuration having a plurality of struts comprising sharp edges;
expanding the apparatus within the access hole to create the cavity;
rotating the apparatus within the cavity sufficiently to cut bone within the cavity; and
contracting the apparatus to facilitate removal of the apparatus from the cavity.

43. The method of claim 42, further comprising removing the cut bone from the cavity.

44. An apparatus for creating a cavity within a bone, comprising:
an expandable basket having a plurality of struts; wherein said struts have sharp edges configured for cutting through bone.

45. An apparatus for creating a cavity within a bone, comprising:
an expandable basket having a plurality of struts; wherein the struts have variable dimensions transverse to the long axis of the struts.

46. The apparatus of claim 45, wherein at least some of the struts are wider in a middle region of the struts than at end regions of the struts.

47. An apparatus for creating a cavity within a bone, comprising:
an expandable basket having a plurality of struts having a first proximal end and a second distal end;
a first substantially rigid member connected to or in contact with a first proximal end of said basket and a second substantially rigid member connected to or in contact with a second distal end of said basket; and
an assembly comprising a screw, said assembly being suitable to exert an axial force on one of said members with respect to the other of said members.

48. The apparatus of claim 47, wherein the screw assembly comprises a knob configured to rotate with respect to a threaded non-rotatable component.

49. The apparatus of claim 47, wherein said screw assembly receives rotation from a translational member.

50. An apparatus for creating a cavity in a bone, comprising:
an expandable basket having a plurality of struts having a proximal end and a distal end; and
an endcap suitable to transmit reaction force to the basket, wherein the endcap has an end which is substantially blunt.

51. The apparatus of claim 50, wherein the endcap further comprises a shoulder region configured for the basket to bear against.

52. An apparatus for creating a cavity within a bone, comprising:
an expandable basket having a plurality of struts having a first end and a second end;
an endcap suitable to transmit force to said basket,
wherein said endcap has a diameter that is greater than the outside diameter of the basket.

53. The apparatus of claim 52, wherein the endcap has a diameter that is less than half of the outside diameter of the endcap.

54. An apparatus for creating a cavity, comprising:
an expandable basket having a plurality of struts;
a flexible membranous covering configured to move outward when the struts move outward;
wherein, in an undeployed configuration, the membranous covering is folded inward to create a space configured to contain particles.

55. The apparatus of claim 54, wherein said particles comprise material which are at least one of osteoconductive or osteoinductive.

56. The apparatus of claim 54, wherein the membranous covering is continuous around a circumference of the apparatus.

57. The apparatus of claim 54, wherein the membranous covering is interspersed around a circumference of the apparatus.

58. The apparatus of claim 54, further comprising an outer membranous covering configured to either rupture or dissolve.

59. The apparatus of claim 54, further comprising an outer membranous covering which comprises holes or slits.

60. The apparatus of claim 54, wherein the plurality of struts comprise spaces configured to contain particles.

61. An apparatus for creating a cavity, comprising:
an elastomer which expands in a radial direction when said elastomer is axially compressed; and
means for axially compressing said elastomer.

62. The method of claim 61, wherein the first particles are radioopaque and the second particles are not radioopaque.

63. A method for treating or preventing a vertebral compression fracture comprising the steps of:
inserting an insertion device percutaneously into a vertebral body;
inserting a cavity-forming device through the insertion device into an area of cancellous bone in the vertebral body;
displacing cancellous bone with the cavity-forming device to create a cavity defined by a surface of cancellous bone;
introducing a first media into the cavity to line at least a portion of the surface thereby reducing the volume of the cavity; and filling at least a portion of the cavity with a second media.

64. A method for treating or preventing a vertebral compression fracture comprising the steps of:
inserting an insertion device percutaneously into a vertebral body;
inserting a cavity-forming device through the insertion device into an area of cancellous bone in the vertebral body;
displacing cancellous bone with the cavity-forming device to create a cavity defined by a surface of cancellous bone;
introducing a first media into the cavity to line at least a portion of the surface thereby reducing the volume of the cavity; and filling at least a portion of the cavity with a second media.

65. The method of claim 64, wherein the insertion device comprises a needle.

66. The method of claim 65, wherein the needle is an eleven-gauge needle.

67. The method of claim 64, wherein the cavity-forming device is selected from the group consisting of: a mechanical tamp, a reamer, a drill, a hole puncher, and a balloon catheter.

68. The method of claim 64 wherein the cavity-forming device is a balloon catheter.

69. The method of claim 64 wherein introducing a first media step comprises introducing a powder into the cavity.

70. The method of claim 64, wherein introducing a first media step comprises introducing a paste into the cavity.

71. The method of claim 64, wherein introducing a first media step comprises introducing a suspension into the cavity.

72. A method of treating a bone, comprising the steps of:
creating a cavity within cancellous bone;
lining at least a portion of the cavity with a first media; and
filling at least a portion of the cavity with a second media;
wherein the first media comprises a cancellous bone ingrowth characteristic and the second media comprises a crack propagation inhibitor characteristic.

73. A method of treating or preventing vertebral compression fracture, comprising the steps of:
creating a cavity within cancellous bone of a vertebral body;
lining at least a portion of the cavity with a first media; and
filling at least a portion of the cavity with a second media to form a construct having an outer surface and a core;
wherein the material of the outer surface comprises a relatively greater bone ingrowth characteristic than the material of the core.

74. A method of treating or preventing vertebral compression fracture as in claim 73, wherein the material of the core has a relatively greater resistance to crack propagation than the material of the outer surface.

75. A kit for treatment of a vertebral compression fracture, the kit comprising:
a cavity forming device;
a first deployment device for deploying a first media against a wall of the cavity; and
a second deployment device for deploying a second media adjacent the first media within the cavity.

76. A kit as in claim 75, additionally comprising a volume of the first media.

77. A kit as in claim 75, additionally comprising a volume of the second media.

78. A kit as in claim 77, wherein the second media comprises PMMA.

79. A kit as in claim 75, additionally comprising access tools for creating access to a vertebral body.
